(19) United States
(12) Patent Application Publication
(10) Pub. No.: US 2006/0079445 A1
(43) Pub. Date: Apr. 13, 2006

(54) CaMKII/calcium channel binding-related compositions and methods

(76) Inventors: Geoffrey S. Pitt, Rye, NY (US); Andy Hudmon, North Haven, CT (US); Howard Schulman, Palo Alto, CA (US)

Correspondence Address: COOPER & DUNHAM, LLP, 1185 AVENUE OF THE AMERICAS, NEW YORK, NY, 10036, US

(21) Appl. No.: 11/155,164

(22) Filed: Jun. 16, 2005

Related U.S. Application Data
(60) Provisional application No. 60/580,570, filed on Jun. 16, 2004.

(51) Int. Cl. A61K 38/17; A61K 38/08; C07K 14/705; C07K 7/06; G01N 33/567

(52) U.S. Cl. 514/2; 514/17; 435/7.2; 530/350; 530/329

(57) ABSTRACT

This invention provides a method for determining whether an agent inhibits binding between CaMKII and a Ca⁺² channel. Furthermore, this invention provides a method for inhibiting binding between CaMKII and a Ca⁺² channel in a cell. Finally, this invention provides methods of treating cardiac arrhythmia and neurological disorders.

This application claims the benefit of U.S. Provisional Application No. 60/580,570, filed Jun. 16, 2004, the contents of which are hereby incorporated by reference.

This invention was made with support under United States Government NIH Grants K08 HL03743, GM30179, and GM40600. Accordingly, the United States Government has certain rights in this invention.

Throughout this application, various publications are referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

Ca²⁺-dependent facilitation (CDF) of calcium channels serves to potentiate the Ca²⁺ influx through the L-type and P/Q type channels during repeated activity. CDF is a feed-forward form of adaptive plasticity that is a critical regulatory feature of many excitable cells. In the heart, frequency-dependent potentiation of Ca²⁺ current through L-type channels (Caᵥ1.2) (Lee, 1987; Marban and Tsien, 1982; Noble and Shimoni, 1981; Schouten and Morad, 1989; Zygmunt and Maylie, 1990) contributes to the force-frequency relationship of cardiac contraction (Koch-Weser and Blinks, 1963). This increased contraction strength with faster heart rates contributes to the positive inotropic response during exercise (Ross et al., 1995) and is abnormal in heart failure (Feldman et al., 1988; Hasenfuss et al., 1994; Mulieri et al., 1992). In brain, CDF of P/Q-type channels (Caᵥ2.1) may contribute to short-term synaptic plasticity (Borst and Sakmann, 1998; Cuttle et al., 1998). In brain, CDF of L-type channels may be important in relation to the privileged role of L-type channels in excitation-transcription coupling (Deisseroth et al., 2003). Thus, understanding the molecular mechanism of CDF would provide a key perspective on the activity-dependent alterations in Ca²⁺ channel function that are central to cardiac function and neuronal plasticity. Despite these important physiological roles that are central to cardiac function and neuronal plasticity, there is little understanding of the molecular mechanism of CDF of L-type channels.

Ca²⁺/calmodulin-dependent protein kinase. II (CaMKII), a multifunctional Ser/Thr protein kinase, is a likely effector of CDF. Pharmacological inhibition of CaMKII abolishes CDF in the heart (Xiao et al., 1994; Yuan and Bers, 1994). Addition of activated CaMKII to the cytoplasmic face of cardiac myocyte membranes induces a high open-probability state of the channel that is consistent with the properties of Ca²⁺ channels displaying CDF (Dzhura et al., 2000). Further, immunocytochemical data suggest that the Caᵥ1.2 and CaMKII are localized close to each other on the cardiomyocyte sarcolemmal membrane (Xiao et al., 1994), suggesting that the kinase has easy access to the channel.

CaMKII has structural and functional properties that make it an ideal candidate to sense the frequency of Ca²⁺ transients during neuronal firing or changes in cardiac rhythm and translate that frequency signal into activity-dependent alterations such as CDF. CaMKII is a multimeric holoenzyme composed of 12 subunits, the subunit isoforms being derived from a family of four closely related genes (α, β, γ, and δ) (Hudmon and Schulman, 2002b) (see, e.g. U.S. Pat. No. 6,518,245). In the brain, α-CaMKII has been shown to play a key role in synaptic plasticity and learning/memory (Lisman et al., 2002). The γ and δ isoforms predominate in the heart and have been implicated in the regulation of gene-expression as well as CDF (Zhang et al., 2002). In all of these isoforms, activation proceeds by Ca²⁺/CaM binding to an autoregulatory region, which causes the removal of a pseudo-substrate domain from the catalytic site. Following the initial stimulus, autophosphorylation of Thr²⁸⁶ or its equivalent (Thr²⁸⁷ in non-α isoforms) renders subsequent kinase activity independent (autonomous) of Ca²⁺ and CaM (Miller et al., 1988) and increases the kinase's affinity for CaM by over 10,000-fold (“CaM trapping”) (Meyer et al., 1992). These properties endow CaMKII with the ability to become persistently activated, in a transition that is sharply dependent on the frequency of Ca²⁺ oscillations (Bayer et al., 2002; Bradshaw et al., 2003; De Koninck and Schulman, 1998).

### SUMMARY OF THE INVENTION

This invention provides a method for determining whether an agent inhibits binding between CaMKII and a calcium channel, comprising: (a) contacting (i) CaMKII, (ii) the calcium channel or a fragment thereof comprising a portion whose amino acid sequence is TVGKF(Y/I) and (iii) the agent, under conditions which, in the absence of the agent, permit binding between CaMKII and the channel or fragment thereof; (b) determining the amount of binding between CaMKII and the channel or fragment thereof in step (a); and (c) comparing the amount of binding determined in step (b) with the amount of binding between CaMKII and the channel or fragment thereof in the absence of the agent, whereby a lower amount of binding in the presence of the agent indicates that the agent inhibits binding between CaMKII and the calcium channel.

This invention also provides an isolated polypeptide comprising a fragment of a calcium channel comprising a portion whose amino acid sequence is TVGKF(Y/I).

This invention further provides a nucleic acid encoding a polypeptide comprising a fragment of a calcium channel comprising a portion whose amino acid sequence is TVGKF(Y/I).

This invention further provides a composition comprising a pharmaceutically acceptable carrier and an agent that inhibits binding between CaMKII and an L-Type and/or P/Q-Type calcium channel.

This invention further provides a method for inhibiting binding between CaMKII and a calcium channel in a cell comprising contacting the cell with an agent that inhibits binding between CaMKII and an L-Type and/or P/Q-Type calcium channel.

This invention further provides a method for inhibiting binding between CaMKII and a calcium channel in a cell comprising contacting the cell with a polypeptide comprising a fragment of an L-Type calcium channel comprising a portion whose amino acid sequence is TVGKF(Y/I).

This invention further provides a method for treating a subject afflicted with cardiac arrhythmia comprising administering to the subject a therapeutically effective amount of an agent that inhibits binding between CaMKII and a calcium channel.

This invention further provides a method for treating a subject afflicted with cardiac arrhythmia comprising administering to the subject a therapeutically effective amount of a polypeptide comprising a fragment of a calcium channel comprising a portion whose amino acid sequence is TVGKF(Y/I).

This invention further provides a method for treating a subject afflicted with (i) epilepsy, (ii) stroke or (iii) a neurodegenerative disorder comprising administering to the subject a therapeutically effective amount of an agent that inhibits binding between CaMKII and a calcium channel.

Finally, this invention provides a method for treating a subject afflicted with (i) epilepsy, (ii) stroke or (iii) a neurodegenerative disorder comprising administering to the subject a therapeutically effective amount of a polypeptide comprising a fragment of a calcium channel comprising a portion whose amino acid sequence is TVGKF(Y/I).

### BRIEF DESCRIPTION OF THE FIGURES

FIGS. 1A-1D: Ca²⁺-dependent facilitation of neuronal L-type current requires CaMKII activity

(A) Representative L-type Ca²⁺ currents in a cerebellar granule cell, elicited by a train of depolarizations to 0 mV from a holding potential of −90 mV. Current shown is the diltiazem-sensitive component of the whole cell Ca²⁺ current (100 μM diltiazem, see Experimental Procedures). The vertical calibration bar represents 10 pA. (B) Same as (A) except with Ba²⁺ as the charge carrier. The vertical calibration bar represents 50 pA. (C) Same as (A) except that the CaMKII inhibitory peptide AIP (2 μM) was included in the intracellular electrode. The vertical calibration bar represents 50 pA. (D) Pooled data representing the calcium current data in the presence or absence of AIP. Current amplitudes for each cell were normalized to the current amplitude elicited by the first pulse. No peptide, n=5; AIP, n=4. The asterisks indicate statistical significance (P<0.05).

FIGS. 2A-2C: Phosphorylation of the α_{1C} subunit by CaMKII

(A) Schematic of α_{1C}. Thick lines highlight regions used to generate GST-fusion proteins. (B) GST-fusion proteins enriched from bacterial lysates using glutathione-sepharose were incubated with purified α-CaMKII in the presence of Ca²⁺/CaM and Mg²⁺/ATP³² as described in the Methods. After extensive washes, proteins were eluted using SDS-PAGE sample buffer. Autoradiogram of fusion proteins separated by SDS-PAGE after phosphorylation by CaMKII. C-term refers to the more distal C-terminal fusion protein containing amino acids 1669-2171. Above the autoradiogram is the Coomassie blue stained band for each fusion protein, indicating equal loading of substrate for all fusion proteins. (C) Autoradiogram showing phosphorylation of α_{1C} by CaMKII. Lysates from HEK cells transfected with α_{1C} and β2b (lanes 2-4) or non-transfected cells (lane 1) were immunoprecipitated with an anti-α_{1C} antibody (lanes 1, 3, and 4) or control IgG (lane 2) and then incubated with purified α-CaMKII in the presence of Ca²⁺/CaM and Mg²⁺/ATP³² as described in the Methods. The CaMKII inhibitor peptide AIP-2 (CalBiochem, 200 nM) was included (lane 4) to demonstrate kinase specificity. Phosphorylated α_{1C} is indicated by arrowhead; autophosphorylated CaMKII, retained after the kinase reaction despite extensive washing of the immunoprecipitate, is indicated with a double arrowhead. An anti-α_{1C} immunoblot of the samples used in the kinase reaction is shown below the autoradiogram.

FIGS. 3A-3C: CaMKII co-immunoprecipitates and co-localizes with α_{1C}

(A) Anti-CaMKII immunoblot of whole brain lysates (Lysate) or following immunoprecipitation (IP) with an anti-α_{1C} antibody. Purified α-CaMKII was run (Control) to demonstrate where CaMKII migrates on the 10% poly-acrylamide gel. The IgG heavy chain (IgG), which migrates just above the ˜50 kDa CaMKII, is denoted by an arrow. A biotinylated CaM overlay was also performed (below, left) to confirm that the band partially obscured by the IgG heavy chain was CaMKII (B) Anti-GFP immunoblot following immunoprecipitation of GFP-CaMKII by anti-α_{1C} antibody (lane 4) or control IgG (lane 5) from lysates of HEK293 cells transiently transfected with GFP-CaMKII and α_{1C}. CaMKII expression in lysates in the absence and presence of α_{1C} (left 3 lanes). Arrow denotes autophosphorylated GFP-CaMKII. (C) Biotinylated calmodulin overlay of rat cardiac sarcolemmal membranes after immunoprecipitation with an anti-α_{1C} antibody. Purified α-CaMKII was run as a control to demonstrate effectiveness of CaM overlay. An anti-α_{1C} antibody (but not control IgG) co-immunoprecipitated a protein identified as the □ isoform CaMKII by biotinylated CaM overlay and apparent molecular weight.

FIG. 4: Activity-dependent interaction of CaMKII with the cytoplasmic determinants of α_{1C}

Immunoblots using a monoclonal antibody (CBα2) for CaMKII after a GST-pull down assay with 20 nM of native (top), Ca²⁺/CaM-activated (middle), or Ca²⁺/CaM/autophosphorylated α-CaMKII (bottom). GST fusion proteins contained various cytoplasmic regions of α_{1C} just as in FIG. 2B. Panel above the immunoblots shows a representative Ponceau stain of each fusion protein. Although only one Ponceau staining profile is shown in FIG. 4, all blots were run in parallel and equal loading of all fusions proteins was independently verified.

FIGS. 5A-5D: Localization of the CaMKII binding site on the C-terminus of α_{1C}

(A) Diagram of α_{1C} and α_{1A} fusion proteins used in GST pull down assays with autophosphorylated α-CaMKII, exhibiting robust (+), partial (∓) and no binding (−). (B) Immunoblot with CBα2 after GST pull down of purified autophosphorylated α-CaMKII (20 nM), using α_{1C} amino acids 1581-1690 fused to GST. Pull down assay performed in the presence of 40 μM of the indicated peptide or the peptide diluent DMSO. (C) Immunoblots with CBα2 or an anti-CaM antibody after a GST pull down assay, using α_{1C} amino acids 1581-1690 (WT), a ¹⁶⁴⁴TVGKFY¹⁶⁴⁹→EEDAAA mutant (Mut6), or GST alone, of purified autophosphorylated CaMKII (left) or CaM (right). Top panels show a Ponceau stain of each fusion protein. (D) Quantification after immunoblot with CBα2 of GST pull down assays of purified autophosphorylated α-CaMKII, using α_{1C} amino acids 1581-1690 (WT), a ¹⁶⁴⁴TVGKFY¹⁶⁴⁹→EEDAAA mutant (Mut6), or GST alone shows that mut6 blocks CaMKII binding. Asterisk indicates p<0.001 for a one-way analysis of variance followed by a Dunnett's test to identify specific pair-wise differences between the means for Mut6 vs. wt and GST vs. wt, n=4-8. Inset shows an exemplar immunoblot with CBα2. Top panels show a Ponceau stain of each fusion protein.

FIGS. 6A-6D: CaMKII interaction with the C-terminus of α_{1C} is essential for CDF

(A) I_{Ba} and scaled I_{Ca} traces during a train of 40 test pulses of Vₕ from −90 mV to +20 mV at 3.3 Hz recorded from oocytes expressing α_{1C} I1654A (I/A) or α_{1C} 1654A/¹⁶⁴⁴TVGKFY¹⁶⁴⁹→EEDAAA (I/A-Mut6). (B) Peak I_{Ba} and I_{Ca} during trains of 40 repetitive test-pulses at 3.3 Hz, normalized to the current amplitude at the beginning of each train (n=4-5). (C) Changes in peak I_{Ba} and I_{Ca} conducted by α_{1C} I1654A (I/A) or α_{1C} I1654A/¹⁶⁴⁴TVGKFY¹⁶⁴⁹→EEDAAA (I/A-Mut6) at indicated stimulation frequencies (n=4-5). (D) Summary of the recovery from inactivation following a two-step protocol for I/A and I/A-Mut6. The length of the pre-pulse was individually determined for each oocyte to produce ˜75-90% inactivation.

FIGS. 7A-7C: The binding site for the C-terminus of α_{1C} on CaMKII is localized near the catalytic domain

(A) Biotinylated CaM overlay of GST-pull downs, using a fusion protein from the C-terminus of α_{1C} (aa 1509-1905) on lysates of HEK293 cells transiently transfected with the CaMKII isoforms (α, β, δ_{A}, δC, and γ_{B}) (arrows) following thio-autophosphorylation. In two rightmost lanes, lysates of untransfected cells were run with (+) and without (−) purified thio-phosphorylated α-CaMKII added to the lysate. (B) Immunoblot using a monoclonal antibody (CBα2) for CaMKII after GST-pull downs, using a fusion protein from the C-terminus of α_{1c} (aa 1509-1905) and purified autophosphorylated α-CaMKII (20 nM). In addition, 20 μM of the indicated peptide was added to each binding reaction. (C) Sequence alignment of CaMKII binding sites from the C-termini of NR2B and α_{1C} with the autoregulatory domain from α-CaMKII.

FIGS. 8A-8E: CaMKII interaction with the C-terminus of α_{1C} is not reversed by dephosphorylation or CaM dissociation and tethered CaMKII requires autophosphorylation or Ca²⁺/CaM for activity

Immunoblots with CBα2 or a phospho-specific CaMKII monoclonal antibody after GST pull down assays, using α_{1C} amino acids 1509-1950 and 20 nM autophosphorylated α-CaMKII. (A) 5 mM EGTA was present in the binding reaction and/or in the wash; in (B), purified recombinant PP1 was added before (PP1-Pre) or after (PP1-Post) the binding reaction in the presence or absence of 5 mM EGTA, as indicated. (C) Time course of reversal of CaMKII autonomous activity following PP1 treatment in solution (n=4). (D) Activity measurements, using peptide AC-2 as a substrate, of CaMKII recovered in GST pull down assays, using α_{1C} amino acids 1509-1950. Ca²⁺/CaM-dependent and autonomous activity measurements of CaMKII recovered after treatment with recombinant PP1 for 30 min (PP1) or no treatment (−) in the binding assay (n=4). (E) Proposed mechanism of CaMKII binding to α_{1C} to form a local and dedicated Ca²⁺ spike integrator for CDF. A catalytic core and autoregulatory domain for a prototypical CaMKII inactive subunit is shown on the lower left (inactive is indicated by green). Ca²⁺/CaM activation and Thr²⁸⁶ autophosphorylation displace the CaMKII autoregulatory domain within the catalytic lobe to activate the subunit (yellow) and to expose an α_{1C} tethering site. The CaMKII holoenzyme remains bound to the α_{1C} C-terminus even after removal of the Ca²⁺/CaM stimulus and CaMKII dephosphorylation produces an inactive subunit. CaMKII may remain tethered to other cytoplasmic domains of α_{1C}, as well. High depolarization frequencies would produce a “threshold” level of activated/autophosphorylated CaMKII subunits that increase the open probability (Po) of the channel via phosphorylation of the N and/or C-termini (upper left). At low depolarization frequencies and under the influence of phosphatase action, CaMKII activation would not be produced, favoring a low Po for α_{1C} (upper right)

### DETAILED DESCRIPTION OF THE INVENTION

### DEFINITIONS

As used in this application, except as otherwise expressly provided herein, each of the following terms shall have the meaning set forth below.

As used herein, “administering” shall mean delivering in a manner which is effected or performed using any of the various methods and delivery systems known to those skilled in the art. Administering can be performed, for example, intravenously, orally, via implant, transmucosally, transdermally, intramuscularly, or subcutaneously. “Administering” can also be performed, for example, once, a plurality of times, and/or over one or more extended periods.

As used herein, “agent” shall include, without limitation, an organic compound, a nucleic acid, a polypeptide, a lipid, and a carbohydrate. Agents include, for example, agents which are known with respect to structure and/or function, and those which are not known with respect to structure or function.

As used herein, “calcium channel” includes, without limitation, an L-type and a P/Q-type calcium channel.

As used herein, “CaMKII” shall mean calcium/calmodulin dependent kinase type II.

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “pharmaceutically acceptable carriers” include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Polypeptide”, “peptide” and “protein” are used equivalently, and each means a polymer of amino acid residues. The amino acid residues can be naturally occurring or chemical analogues thereof. Polypeptides and proteins can also include modifications such as glycosylation, lipid attachment, sulfation, hydroxylation, and ADP-ribosylation.

As used herein, “subject” shall mean any animal, such as a primate, mouse, rat, guinea pig or rabbit. In the preferred embodiment, the subject is a human.

As used herein, “treating” a subject afflicted with a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

### EMBODIMENTS OF THE INVENTION

This invention provides a method for determining whether an agent inhibits binding between CaMKII and a calcium channel, comprising: (a) contacting (i) CaMKII, (ii) the calcium channel or a fragment thereof comprising a portion whose amino acid sequence is TVGKF(Y/I) and (iii) the agent, under conditions which, in the absence of the agent, permit binding between CaMKII and the channel or fragment thereof; (b) determining the amount of binding between CaMKII and the channel or fragment thereof in step (a); and (c) comparing the amount of binding determined in step (b) with the amount of binding between CaMKII and the channel or fragment thereof in the absence of the agent, whereby a lower amount of binding in the presence of the agent indicates that the agent inhibits binding between CaMKII and the calcium channel.

In one embodiment of the instant method, the calcium channel is an L-Type calcium channel. In another embodiment, the calcium channel is a P/Q-type calcium channel.

In one embodiment, the CaMKII of step (a) is autophosphorylated. In another embodiment, a fragment of the calcium channel is used. In one embodiment, the fragment of calcium channel comprises a portion whose amino acid sequence is TVGKFY. In another embodiment, the fragment of calcium channel is of an L-type calcium channel and comprises a portion whose amino acid sequence is that of the portion of the channel from amino acid reside 1639 to amino acid residue 1660. In another embodiment, the fragment of calcium channel is at least about 10, 20 or 50 amino acid residues in length.

In one embodiment, the agent can be a known kinase inhibitor or a polypeptide. This polypeptide may comprise a fragment of calcium channel comprising a portion whose amino acid sequence is TVGKF(Y/I). This polypeptide may further comprise a fragment of calcium channel comprising a portion whose amino acid sequence is TVGKFY. The polypeptide may also comprise a fragment of L-type calcium channel comprising a portion whose amino acid sequence is that of the portion of the channel from amino acid reside 1639 to amino acid residue 1660. The polypeptide may also comprise a fragment of calcium channel and is at least about 10, 20 or 50 amino acid residues in length.

This invention further provides an isolated polypeptide comprising a fragment of a calcium channel comprising a portion whose amino acid sequence is TVGKF(Y/I).

In one embodiment of the polypeptide, the fragment of calcium channel comprises a portion whose amino acid sequence is TVGKFY. In other embodiments, the calcium channel is an L-Type calcium channel or a P/Q-type calcium channel. In another embodiment, the fragment is of L-Type calcium channel and comprises a portion whose amino acid sequence is that of the portion of the channel from amino acid reside 1639 to amino acid residue 1660. In another embodiment, the fragment of calcium channel is at least about 10, 20 or 50 amino acid residues in length.

This invention further provides a nucleic acid encoding a polypeptide comprising a fragment of a calcium channel comprising a portion whose amino acid sequence is TVGKF(Y/I).

In embodiments of the nucleic acid, the calcium channel is an L-Type calcium channel or a P/Q-type calcium channel.

This invention further provides a composition comprising a pharmaceutically acceptable carrier and an agent that inhibits binding between CaMKII and an L-Type and/or P/Q-Type calcium channel.

This invention further provides a method for inhibiting binding between CaMKII and a calcium channel in a cell (e.g. a human cell) comprising contacting the cell with an agent (e.g. AIP-2, AC-2 and AC-3I) that inhibits binding between CaMKII and an L-Type and/or P/Q-Type calcium channel. In one embodiment, the calcium channel is an L-type channel and the cell is a cardiac cell (e.g. a human cardiac cell). In another embodiment, the cell is a neuronal cell (e.g. a human neuronal cell).

This invention further provides a method for inhibiting binding between CaMKII and a calcium channel in a cell comprising contacting the cell with a polypeptide comprising a fragment of an L-Type calcium channel comprising a portion whose amino acid sequence is TVGKF(Y/I). In one embodiment, the calcium channel is an L-type channel and the cell is a cardiac cell (e.g. a human cardiac cell). In another embodiment, the cell is a neuronal cell (e.g. a human neuronal cell).

This invention further provides a method for treating a subject afflicted with cardiac arrhythmia comprising administering to the subject a therapeutically effective amount of an agent that inhibits binding between CaMKII and a calcium channel. In one embodiment, the subject is human. In another embodiment, the calcium channel is an L-type calcium channel.

This invention further provides a method for treating a subject afflicted with cardiac arrhythmia comprising administering to the subject a therapeutically effective amount of a polypeptide comprising a fragment of a calcium channel comprising a portion whose amino acid sequence is TVGKF(Y/I). In one embodiment, the subject is human.

This invention further provides a method for treating a subject afflicted with (i) epilepsy, (ii) stroke or (iii) a neurodegenerative disorder comprising administering to the subject a therapeutically effective amount of an agent that inhibits binding between CaMKII and a calcium channel. In one embodiment, the subject is a human. In other embodiments, the calcium channel is an L-type calcium channel or a P/Q-type calcium channel. In another embodiment, the subject is afflicted with epilepsy. In other embodiments, the subject is afflicted with a neurological disorder such as Alzheimer's disease (where excessive calcium leads to neurodegeneration) (see, e.g. Tymianksi 2003; Mattson 2004) or stroke (where it is advantageous to limit further damage due to glutamate stimulation).

Finally, this invention provides a method for treating a subject afflicted with (i) epilepsy, (ii) stroke or (iii) a neurodegenerative disorder comprising administering to the subject a therapeutically effective amount of a polypeptide comprising a fragment of a calcium channel comprising a portion whose amino acid sequence is TVGKF(Y/I). In one embodiment, the subject is a human. In other embodiments, the calcium channel is an L-type calcium channel or a P/Q-type calcium channel. In another embodiment, the subject is afflicted with epilepsy. In other embodiments, the subject is afflicted with a neurological disorder such as Alzheimer's disease, or stroke.

In one embodiment, dosage is between 1 mg and 200 mg per human subject, or weight equivalent thereof for a non-human subject. In a further embodiment, dosage is between 5 mg and 50 mg per human subject, or weight equivalent thereof for a non-human subject. In a further embodiment, dosage is between 10 mg and 20 mg per human subject, or weight equivalent thereof for a non-human subject.

One category of conditions that may be treated or modulated by using the subject methods are conditions associated with CDF in cardiac cells. As such, the subject methods may be employed in the treatment or modulation of cardiac tissue related conditions, e.g., in treating arrhythmia and protection from arrhythimia due to certain antiarrythmic drugs, hypertrophy, high blood pressure, and other conditions associated with excessive muscular contraction. Another category of conditions that may be treated or modulated by using the subject methods are conditions associated with CDF in neuronal cells. As such, the subject methods may be employed in the treatment or modulation of neuronal tissue related conditions, including brain tissue related conditions, e.g., in promoting learning, memory, and in providing neuroprotection from stroke, ischemia or epilepsy. The instant methods can be applied, as appropriate, to single cells or in vivo.

This invention is illustrated in the Experimental Details section which follows. This section is set forth to aid in an understanding of the invention but is not intended to, and should not be construed to, limit in any way the invention as set forth in the claims which follow thereafter.

Experimental Details

Synopsis

Ca²⁺-dependent facilitation of voltage-gated calcium current (CDF) is a powerful mechanism for upregulation of Ca²⁺ influx during repeated membrane depolarization. CDF of L-type Ca²⁺ channels was found in cerebellar granule neurons and could be prevented by a peptide inhibitor of CaMKII, in line with previous findings in heart cells. CDF of L-type Ca²⁺ channels (Caᵥ1.2) contributes to the positive force-frequency effect in the heart and is believed to involve the activation of Ca²⁺/calmodulin-depenent kinase II (CaMKII). Biochemical analysis demonstrated that CaMKII interaction with the C-terminus of the pore forming subunit α_{1C} (Caᵥα1.2) is essential for CDF of L-type channels. How CaMKII is activated and what its substrate(s) are has not yet been determined. Here it will be shown that the pore forming subunit α_{1C} (Caᵥα1.2) is a CaMKII substrate and that CaMKII interaction with the C-terminus of α_{1C} is essential for CDF of L-type channels. Ca²⁺ influx triggers distinct modes of CaMKII targeting and activity. After Ca²⁺-induced targeting to α_{1C}, CaMKII becomes tightly tethered to the channel, even after calcium returns to normal levels. In contrast, the activity of the CaMKII tethered to its substrate, α_{1C}, retains its Ca²⁺/CaM dependence, explaining its ability to operate as a calcium spike frequency detector. These findings reveal the molecular basis of CDF and demonstrate a novel enzymatic mechanism by which ion channel gating can be modulated by activity.

It is now demonstrated for the first time that the subcellular localization of CaMKII is critical for its activity and biological role as a Ca²⁺ spike frequency decoder. It is shown that CaMKII phosphorylates α_{1C} and that tethering of CaMKII to the α_{1C} C-terminus is an essential molecular step feature of CDF. It is shown that a molecular model for CDF in which a dedicated CaMKII holoenzyme acts as both a local sensor to monitor Ca²⁺ channel activity and as a resident kinase effector to regulate Ca²⁺ channel activity.

Methods

Neuronal Cultures and Transfections

Dissociated cerebellar granule cells were prepared from 7-day old C57/BL6 mouse cerebella and grown in culture by a published protocol (Piedras-Renteria and Tsien, 1998; Randall and Tsien, 1995). The cerebellar granule cells were maintained in Minimal Essential Medium (Invitrogen) supplemented with 10% fetal bovine serum, glucose 5 mg/liter, transferrin 100 mg/liter, insulin 25 mg/liter, glutamine 300 mg/liter, and potassium chloride 20 mM. Cells were plated on Matrigel (Collaborative Biomedical Product) treated glass coverslips in 24-well chambers and maintained in a humidified, 37° C. incubator with 95% O₂/5% CO₂. Cells were maintained for 6 to 9 days in culture prior to electrophysiological experiments. HEK293 cells stably transfected with the α₂-δ and β_{1C} subunits (HEK_{α2β1}) were grown in Dulbecco's Modified Eagle's Medium containing 10% v/v calf serum, penicillin (100 units/ml), streptomycin (100 μg/ml), and kept under positive selection with Geneticin (400 μg/ml) while being maintained as described above. cDNAs encoding the α_{1C77} subunit (Zühlke and Reuter, 1998) and the green fluorescent protein were transiently transfected into HEK_{α2β1} cells by using the Lipofectamine2000 reagent (Invitrogen). Electrophysiological experiments were performed >24 hrs after transfection.

Patch-Clamp Recordings

Cerebellar granule cells, identified on the basis of morphology and size, comprised >90% of the cells in the primary culture. The whole-cell patch-clamp technique (EPC7 amplifier, HEKA) was used to record Ca²⁺ channel activity with Ca²⁺ or Ba²⁺ as the charge carrier. Cells were initially bathed in a solution containing (in mM) NaCl 119, KCl 5, CaCl₂ 2, MgCl₂ 1, glucose 30, HEPES-NaOH 25 (pH 7.3), 305 mOsm. After establishing the whole-cell configuration, the bath solution was replaced by one comprised of (in mM): TEA-Cl 155, CaCl₂ 10 (or BaCl₂ 10), HEPES 10, glucose 10 (pH 7.3 with CsOH), 305 mOsm. In all cases electrodes contained (in mM): CsCl 109, MgCl₂ 4.5, EGTA 1, ATP 4, GTP 0.3, HEPES 25, phosphocreatinine 10, creatine phosphokinase 20 units/mL (pH 7.3 with CsOH), 295 mOsm. The potential difference between the open electrode and the bath ground was zeroed prior to establishing a ≧1 GΩ resistance seal. Currents were low-pass-filtered at 3 kHz and digitized at 10 kHz (Digidata, Axon Instruments Inc) for storage on the hard drive of a Pentium PC. Voltage-gated Ca²⁺ and Ba²⁺ currents were recorded from cells depolarized to 0 mV with five 20 ms pulses (50 ms interpulse interval) from a −90 mV holding potential. L-type Ca²⁺ currents were isolated by subtracting the current remaining after the application of diltiazem (100 μM) (Sigma) from the current prior to the application of diltiazem. Use-dependence of L-type current block by diltiazem has been previously reported (Cai et al., 1997). Accordingly, checks were run for use-dependence of diltiazem block of calcium currents supported by cloned L-type channels (subunit α_{1C77}), using the same solutions and voltage protocol described above, but none were found. For peptide inhibitor experiments, the internal solution contained 2 μM autocamtide-2-related inhibitory peptide (AIP)(amino acid sequence, KKALRRQEAVDAL) (Biomol Research Labs). This internal solution was made on the day of the experiment by dilution of a 500 μM stock solution with normal internal solution.

Oocyte Recordings

The plasmid encoding the rabbit cardiac α_{1C} subunit used for expression in Xenopus oocytes, pCARDHE, was a gift of W. Sather (U. of Colorado). In vitro transcription and microinjection into Xenopus oocytes of α_{1C}, the auxiliary Ca²⁺ channel subunits β₁ and α₂δ were performed as previously described (Zühlke et al., 2000). Before recording whole cell I_{Ba} or I_{Ca}, oocytes were injected with 25-50 nl of 100 mM BAPTA solution (pH 7.4) to minimize contaminating Ca²⁺-activated Cl⁻ currents. I_{Ba} and I_{Ca} recordings were performed essentially as described (Zühlke et al., 2000) with a standard two-electrode voltage clamp configuration using an oocyte clamp OC-725C amplifier (Warner Instrument Corp.) connected through a Digidata 3122A A/D interface (Axon Instruments) to a personal computer. I_{Ba} and I_{Ca} were recorded in the same oocyte. Ionic currents were filtered at 1 kHz by an integral 4 pole Bessel filter and sampled 10 kHz and analyzed with Clampfit 8.1.

GST-Fusion Proteins

PCR fragments corresponding to the α_{1C} (Genbank accession # X15539) N-terminus (aa 1-154), I-II intracellular loop (aa 435-554), II-III intracellular loop (aa 784-931), III-IV intracellular loop (1197-1250), and two C-terminal fragments (aa 1581-1690, and aa 1669-2171) were cloned into pGEX-4T-1 and GST fusion proteins were generated. The plasmids encoding the C-terminal fragments CT5 (aa 1507-1622), CT12 (aa 1509-1905), and CT23 (aa 1622-1905) were kindly provided by M. Hosey (Northwestern Univ.).

Peptides

Peptides spanning α_{1C} residues 1581-1690 have previously been described (Pitt et al., 2001). The NMDA-L peptide (Bayer et al., 2001), peptides AC-2 (Hanson et al., 1989), AC-3i (Braun and Schulman, 1995), and AC-3c (Braun and Schulman, 1995) have been described elsewhere.

Immunocytochemistry

The epitope-tagged brain α_{1C} subunit was a gift from R. Dolmetsch (Harvard Univ.) and detected using an anti-Xpress monoclonal antibody (Invitrogen). Cells were imaged using a NIKON LSM-510 confocal microscope. GFP-CaMKII expressed alone was homogenously distributed throughout the cytoplasm, as previously reported (Bayer et al., 2001). GFP co-expressed with α_{1C} showed no apparent co-localization.

Immunoprecipitation

Whole rat brain lysates were solubilized in 150 mM NaCl, 50 mM Tris, pH 8.0, 1% Triton, and Complete protease inhibitor cocktail (Roche). HEK293 cells expressing α_{1C}, α₂δ, β2, and CaMKII-α were lysed in 150 mM NaCl, 50 mM Tris, pH 8.0, 1% Triton, and Complete protease inhibitor cocktail (Roche). In both cases, the supernatant was pre-cleared and then immunoprecipitated with anti-α_{1C} antibody (Alomone). Rat cardiac sarcolemmal membranes were kindly provided by S. O. Marx (Columbia University). Immunoprecipitation was performed with either an anti-α_{1C} (Alomone) or control IgG in 150 mM NaCl, 50 mM Tris, pH 8.0, 1% Triton, and Complete protease inhibitor cocktail (Roche). After SDS-PAGE, calmodulin overlay was performed with biotin conjugated calmodulin (STI Signal Transduction) and detected with Vectastain ABC kit (Vector laboratories). HEK293 cells were transfected with α_{1C}, α₂δ, β2, and GFP-CaMKII using Lipofectamine 2000 (Invitrogen) as instructed by the manufacturer. After 48 h, they were washed in ice-cold PBS and then lysed in 150 mM NaCl, 50 mM Tris, pH 8.0, 1% Triton, and Complete protease inhibitor cocktail and immunoprecipitation was performed with the anti-α_{1C} antibody (Alamone). After SDS-PAGE, immunoblot was performed with an anti-GFP antibody (Covance).

Expression and Purification of CaMKII

α-CaMKII was expressed and purified essentially as described (Bradshaw et al., 2002). Additional CaMKII isoforms were generated by transient expression in HEK293 cells (Srα plasmid containing the α, β, δ_{A}, δ_{C}, or γ_{B} isoforms). After 72 hrs, cells were lysed in 10 mM Tris/5% Betaine/150 mM sodium perchlorate, pH 7.5 by brief sonication. Cell lysates were centrifuged for 30 min at 14,000×g at 4° C. and the supernatants aliquoted, snap frozen, and stored at −80° C.

GST Binding Assay

The binding reactions were accomplished in Tris binding buffer (50 mM Tris, 150 mM NaCl, 0.1% T-20 at pH 7.4 ml plus 0.1% BSA) containing 20 nM purified CaMKII. The total protein from the HEK293 cell lysates added to each binding reaction ranged from 9-22 μg, determined by normalizing for the amount of CaMKII activity (Singla et al., 2001). Pre-autophosphorylayion of CaMKII (purified and lysate) was accomplished at 4° C. for 5 min in Tris binding buffer plus 1 mM CaCl₂, 5 μM CaM, 1 mM ATP, and 5 mM MgCl₂ to restrict the sites of autophosphorylation to primarily Thr286 (Ikeda et al., 1991; Lai et al., 1987; Lou and Schulman, 1989). Final concentration of these components in the binding reaction (1:40) was 0.025 mM CaCl₂, 0.125 μM CaM, 0.025 mM ATP, and 0.125 MM MgCl₂. The binding reaction was rocked for 1 h at 4° C. and the beads extensively washed in Tris binding buffer (2-3× for 5 mins each). Immunoblots were performed multiple times with identical results and Ponceau staining was always performed to ensure equal loading and transfer. CaMKII binding was quantified using densiotometric measurement of band intensity using KODAK 1D Image Analysis Software (Eastman Kodak, Company). Multiple exposure times, as well as a standard curve generated by dilution analysis, ensured linearity in the chemiluminescence intensity. One-way analysis of variance was performed and a Dunnett's test was used to identify specific pair-wise differences between the means. Comparison analyses were conducted using SPSS Version 10.1.3.

Calmodulin Binding Assay

The bound GST proteins/sepharose complex was prepared as described above. Purified CaM (Singla et al., 2001) was applied in the presence of 1 mM CaCl₂ for 1 h, before multiple washes of Tris binding buffer plus 1 mM CaCl₂. Immunoblotting was performed as described (Pitt et al., 2001).

CaMKII Phosphorylation of α_{1C}

GST-fusion proteins were bound to Glutathione Sepharose as above. CaMKII was pre-autophosphorylated and then added to the bound GST-fusion proteins in the presence of Mg/ATP³² added for 15 min at room temperature. The reaction was terminated with the addition of SDS-PAGE sample buffer. SDS-PAGE and Coomassie staining/destaining was followed by autoradiography. Purified α-CaMKII was incubated with bound GST-fusion proteins or immunoprecipitated material bound to Protein A in the presence of Ca²⁺/CaM (2 mM/10 μM) and Mg²⁺/ATP (5 mM/50 μM ATP) plus 10-50 μCi ATP³² for 15 min at room temperature. For the GST-proteins, CaMKII was activated prior to exposure to the substrate reaction on ice as described above under GST-binding assay to produce autophosphorylated enzyme. After the phosphorylation, the beads were washed extensively in PBS (plus 5 mM EDTA) and 2× SDS-PAGE sample buffer was added and SDS-PAGE performed. The gels were Coomassie stained and exhaustively destained. The gels were dried down and P³²-labeled proteins were detected using autoradiography.

CaMKII Dephosphorylation using PP1

CaMKII was dephosphorylated using a Hisx6-tagged PP1 catalytic subunit construct (gift from Angus Nairn, Yale University) purified by Ni-NTA affinity chromatography.

Results

Ca²⁺-Dependent Facilitation of L-Type Currents in Central Neurons

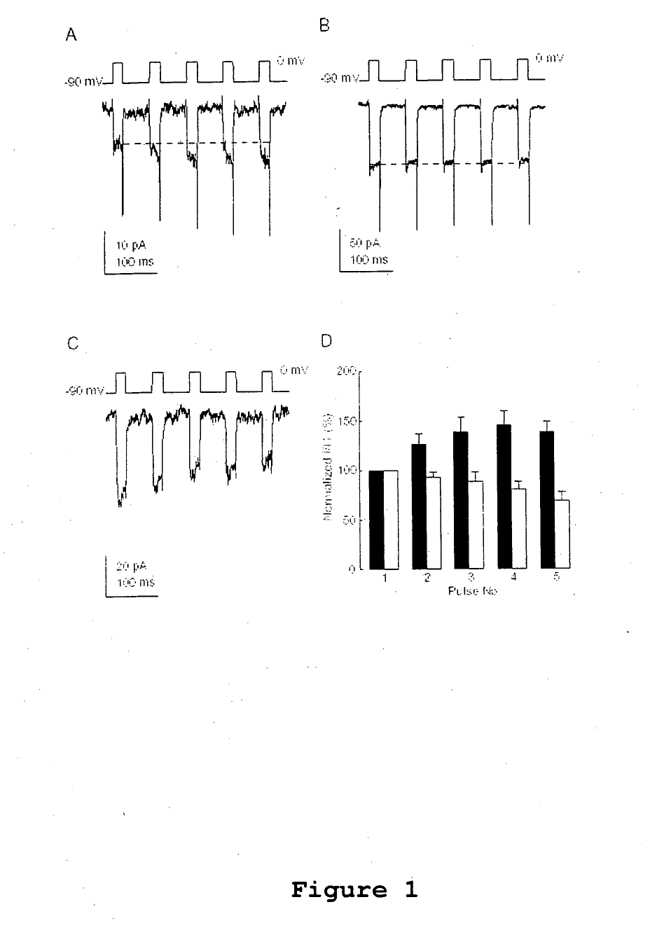

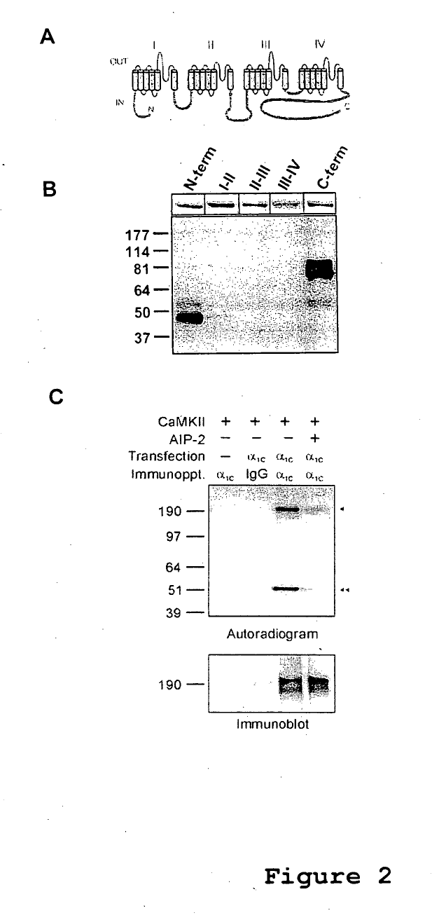

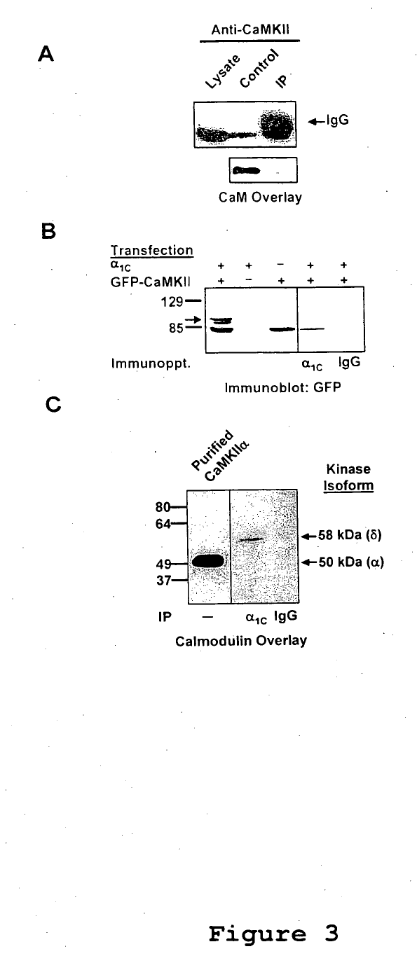

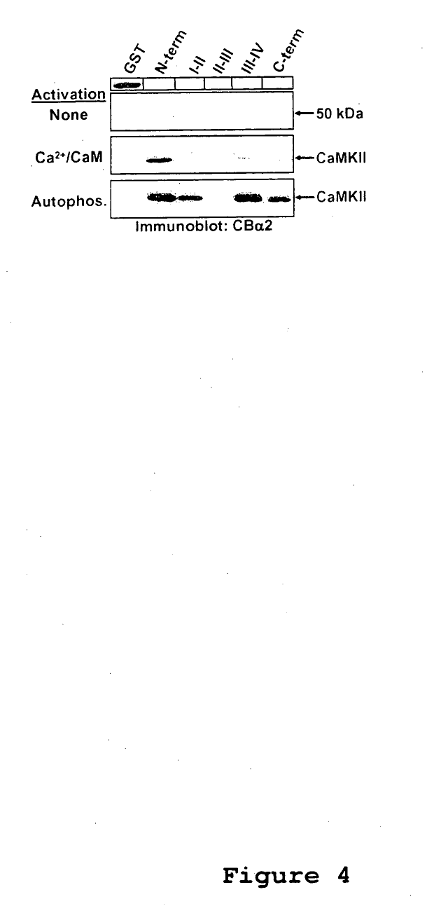

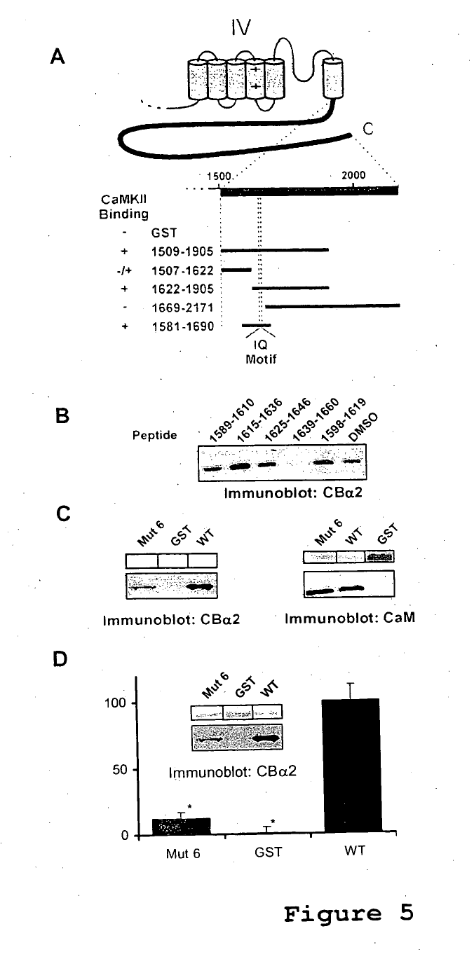

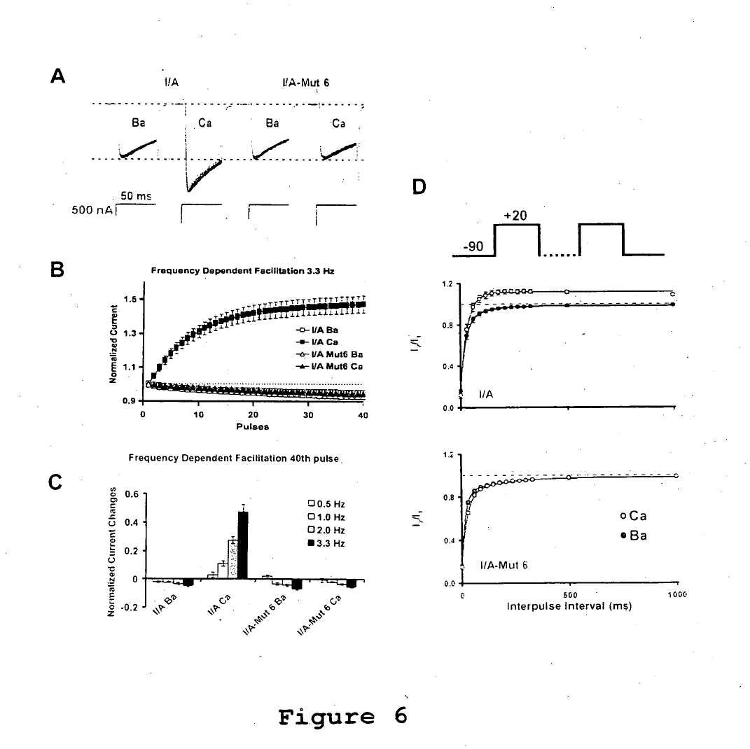

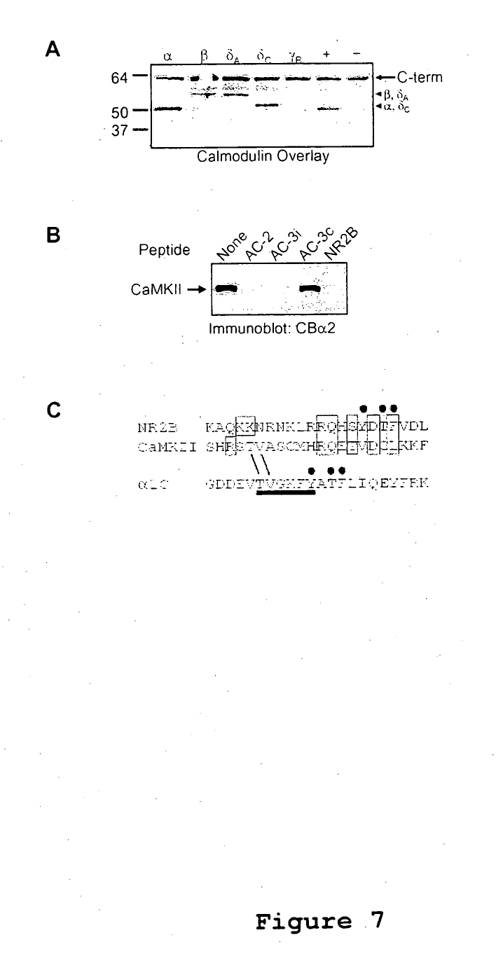

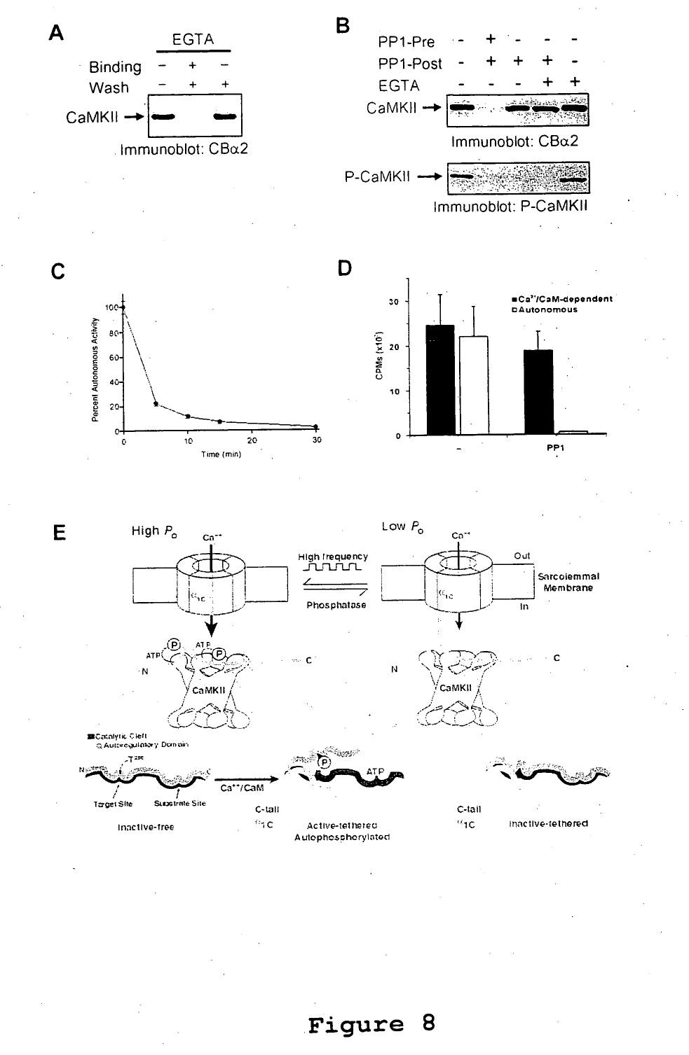

Ca²⁺-dependent facilitation of Ca²⁺ currents has been carefully studied in heart cells but little or no information is available about the existence of CDF of Ca²⁺ channels in a native neuronal system. Patch clamp recordings in cultured cerebellar granule cells were carried out to look for CDF in neurons. Currents carried by L-type channels were evoked by trains of brief depolarizing pulses and pharmacologically dissected using the L-type channel antagonist diltiazem (FIG. 1). With Ca²⁺ as the external charge carrier, L-type currents grew progressively larger in amplitude with successive depolarizations (FIG. 1A). The facilitation averaged ˜50% over the course of a train of five pulses (FIG. 1D). In contrast, no such increase was seen with Ba²⁺ as the external divalent cation (FIG. 1B), consistent with the idea that the facilitation was specifically dependent on the influx of Ca²⁺, as expected for CDF. Previous experiments on CDF in heart cells have demonstrated that Ca²⁺ entry is required because it initiates a signaling cascade involving CaMKII (Hryshko and Bers, 1990; Xiao et al., 1994; Yuan and Bers, 1994). L-type Ca²⁺ currents with an internal solution containing a CaMKII-inhibiting peptide (autocamtide-2-related inhibitory peptide) in the patch pipette were recorded to probe the possible involvement of CaMKII in granule neurons. In this case, the L-type Ca²⁺ current amplitude declined progressively with repeated depolarization, as expected for Ca²⁺-dependent inactivation (CDI) rather than CDF (FIG. 1C). The decrease in peak L-type current averaged ˜25% over the course of the depolarizing pulse train (FIG. 1D). These recordings demonstrated the existence of Ca²⁺-dependent facilitation of L-type currents in cerebellar granule cells, by far the most abundant nerve cells in the CNS. In these neurons, CDF was clearly dependent on Ca²⁺ entry and on the activity of CaMKII, in keeping with previous work on CDF in cardiac myocytes (Anderson et al., 1994; Xiao et al., 1994; Yuan and Bers, 1994). This was as expected if highly similar molecular components (CaM, CaMKII and □_{1C}) were in play in neurons and heart cells.

The N- and C-Terminus of α_{1C} are Substrates of CaMKII

Modulation of L-type channel gating by cytoplasmic delivery of constitutively active CaMKII is blocked by non-hydrolyzable analogs of ATP (Dzhura et al., 2000), suggesting that the kinase acts through phosphorylation of the channel or an associated regulatory protein. Accordingly, tests were run to determine whether CaMKII could directly phosphorylate any of the intracellular domains of α_{1C}, the pore-forming subunit of the channel. Since the kinase-induced increase in L-type Ca²⁺ current by both PKA and Src results from phosphorylation of α_{1C} (Bence-Hanulec et al., 2000; De Jongh et al., 1996), it was first tested whether α_{1C} was also a substrate for activated CaMKII. As shown in FIG. 2C, addition of activated CaMKII to α_{1C} immunoprecipitated from HEK lysates in which L-type channels have been expressed results in phosphorylation of protein migrating at ˜240 kDa, consistent with the M_{w} of α_{1C}. The kinase activity could be attributed to CaMKII and not to another kinase co-immunoprecipitated with α_{1C}, since inclusion of the CaMKII inhibitor AIP-2 prevented phosphorylation; continued presence of the α_{1C} protein under this condition was confirmed by immunoblotting (lower panel). The immunoprecipitated and phosphorylated protein could be confidently identified as α_{1C} in light of the finding that no α_{1C} was immunoprecipitated nor was ³²P incorporated when immunoprecipitation was performed with control IgG or with lysates of HEK cells in which α_{1C} had not been expressed. Interestingly, under conditions in which α_{1C} was phosphorylated by CaMKII (lane 3), a ³²P labeled protein (˜50 kDa) was noticed corresponding to the autophosphorylated form of the α-subunit of CaMKII that had been introduced for the kinase assay. The retention of CaMKII, despite extensive washing of the immobilized α_{1C}, suggested that α_{1C} may serve as an anchoring protein as well as a substrate for the kinase. The lack of retention when AIP-2 was added to the reaction gave an early indication about the mechanism of anchoring (see FIG. 7B).

Having demonstrated that α_{1C} was a CaMKII substrate, the next tests were to determine which of the intracellular domains of α_{1C} were phosphorylated by CaMKII. GST fusion proteins were generated for the entire sequence of each of the intracellular domains of the α_{1C} subunit except the large cytoplasmic tail, which was represented by two complementary fragments (amino acids 1507-1622 and 1669-2171) (FIG. 2A). When the fusion proteins were tested in an in vitro kinase assay, significant incorporation of ³²P was only observed for the N-terminal construct and the C-terminal fusion protein containing amino acids 1669-2171 (FIG. 2B), and not the fusion protein containing aa 1507-1622 (data not shown). The finding that CaMKII can phosphorylate N- and C-terminal regions of α_{1C} is provocative in light of previous data suggesting that these regions may be targets of kinase action for modulation of Caᵥ1.2 function (Bence-Hanulec et al., 2000; McHugh et al., 2000; Rotman et al., 1995). Similar to results with the intact channel (FIG. 2C), a ˜50 kDa ³²P labeled protein corresponding to the autophosphorylated form of the α-subunit of CaMKII that had been introduced for the kinase assay was again noticed in multiple lanes. The finding that α-CaMKII could be retained by individual domains of α_{1C} suggested that these domains might contribute to the kinase anchoring to the channel subunit as a whole. Interestingly, with longer exposures, it was noticed that in multiple lanes a ³²P labeled protein migrating slightly above ˜50 kDa, in all likelihood corresponding to the autophosphorylated form of the α-subunit of CaMKII that had been introduced for the kinase assay. The retention of α-CaMKII was striking because the fusion proteins bound to Glutathione-agarose had been subjected to multiple washes after the phosphorylation reaction in order to remove unincorporated γ-³²P-ATP. This raised the possibility that CaMKII might interact directly with the α_{1C} GST-fusion proteins.

CaMKII Interacts Specifically with α_{1C}

The possibility that CaMKII tethers to α_{1C} in rat heart by attempting to co-immunoprecipitate CaMKII with α_{1C} was tested (FIG. 3C). An anti-α_{1C} antibody (but not control IgG) co-immunoprecipitated a ˜58 kDa protein from rat heart that was easily detectable with a biotinylated calmodulin overlay, consistent with the properties of δ-CaMKII. Tethering of the kinase to the pore-forming subunit was further evaluated in experiments with HEK293 cells co-expressing GFP-tagged CaMKII and Xpress-tagged-α_{1C}, along with the calcium channel accessory subunits α₂δ and β₂ (FIG. 3B). Co-immunoprecipitation of the GFP-CaMKII by the antibody to epitope-tagged α_{1C} (lane 4), but not by a control IgG (lane 5), was observed.

The possibility that CaMKII interacted with α_{1C} by attempting to co-immunoprecipitate CaMKII with α_{1C} from rat brain was tested. As shown in FIG. 3A, an anti-α_{1C} antibody co-immunoprecipitated CaMKII from a lysate of whole rat brain. Because the CaMKII signal on the immunoblot was partially obscured by the signal from the IgG heavy chain of the immunoprecipitating anti-α_{1C} antibody, which migrates slightly above the α-CaMKII, the interaction of CaMKII with α_{1C} by three different approaches was confirmed. First, an overlay with biotinylated CaM was performed. As seen in FIG. 3A, the CaM overlay recognized a band in the immunoprecipitate that migrated identically to purified CaMKII. Additionally, tests were run to co-express GFP-tagged CaMKII with Xpress-tagged-α_{1C}, along with the calcium channel accessory subunits α₂δ and β₂ in HEK293 cells and look for the co-immunoprecipitation of GFP-tagged CaMKII with the epitope-tagged α_{1C} (FIG. 3B) When total lysates of HEK293 cells expressing Xpress-tagged-α_{1C}, α₂δ, β₂, and GFP-α-CaMKII were immunoprecipitated with an anti-Xpress epitope antibody, co-immunoprecipitation of GFP-CaMKII (lane 4) was observed; no co-immunoprecipitation was observed with a control IgG (lane 5).

Additional bands were noted on the immunoblot for GFP-CaMKII (lanes 1-3, FIG. 3B, denoted by an arrow) displaying an electrophoretic mobility shift typical of that observed with autophosphorylation (Hudmon et al., 1996; Lou et al., 1986), especially prominent when □_{1C} was co-expressed. Although faint in the □_{1C} immunoprecipitated GFP-CaMKII (lane 4), these bands were more visible in longer exposures, suggesting that expression of the Ca²⁺ channel somehow promotes or stabilizes the autophosphorylation of the kinase.

Activity-Dependent Association of CaMKII with Multiple Cytoplasmic Regions of α_{1C}

To define further the interaction between CaMKII and α_{1C} and whether different activation states of CaMKII modulated binding, a pull-down binding assay using the various α_{1C}-GST fusion proteins was constructed to test whether a direct interaction in vitro could be observed and whether different activation states of CaMKII modulated binding (FIG. 4). When CaMKII was activated with Ca²⁺/CaM, but not allowed to undergo autophosphorylation, the kinase bound to the N-terminal domain and the III-IV loop of α_{1C} (FIG. 4, middle row). Subsequent removal of Ca²⁺/CaM reversed this binding (data not shown). When CaMKII was activated in the presence of Ca²⁺/CaM plus ATP plus ATP on ice, conditions previously shown to produce predominantly Thr286 autophosphorylation (Ikeda et al., 1991; Lai et al., 1987; Lou and Schulman, 1989), CaMKII was found once more to bind to the N-terminus and III-IV loop, and additionally, to the I-II loop and the C-terminus as well (FIG. 4, bottom row).

In contrast, CaMKII did not bind to any of the cytoplasmic region-containing GST fusion proteins in the absence of activating stimuli (FIG. 4, top row). It was concluded that the initiation of a direct interaction between CaMKII and α_{1C} requires some form of activation of the kinase by Ca²⁺/CaM, binding of Ca²⁺/CaM or autophosphorylation. A subsequent activation state, that produced by autophosphorylation, was necessary for binding to additional cytoplasmic regions of α_{1C}. In addition, depending on the cytoplasmic determinants of α_{1C} assayed, the exposure of the corresponding binding site on CaMKII may require different activation states.

To identify novel structural determinants of α_{1C} that functionally affect Ca²⁺-dependent facilitation, initially the C-terminus was initially focused. This region displays an appropriate combination of attributes for CaMKII-mediated CDF: it is a target for phosphorylation by CaMKII (FIG. 2B), it binds preferentially to autophosphorylated CaMKII (FIG. 4), a state of the kinase capable of supporting facilitation of single channels (Dzhura et al., 2000), and it has been implicated in Ca²⁺-dependent modulation of channel function (for example, see ref. (Gao et al., 2001; Hell et al., 1995; Zühlke et al., 1999)). To delimit the locus of CaMKII binding within the C-terminal tail of α_{1C}, a series of GST fusion proteins corresponding to different portions of this region were used (FIG. 5A). A pattern of interactions with autophosphorylated CaMKII was found that suggested that the kinase bound between residues 1622 and 1669 of α_{1C}. Because a weak interaction was also seen with a construct proximal to 1622, a fusion protein was generated spanning amino acids 1581-1690 for additional testing. To narrow down further the CaMKII interaction site within this 110 amino acid region, its interaction with autophosphorylated CaMKII was probed for interference by a series of overlapping ˜22 aa peptides (Pitt et al., 2001) (FIG. 5B). A peptide generated from residues 1639-1660 dramatically reduced the interaction of the kinase with the 1581-1690 fusion protein. In contrast, the CaMKII interaction was not inhibited by two peptides, 1589-1610 and 1615-1636, that corresponded to sites important for tethering of apoCaM (Kim et al., 2004; Pitt et al., 2001). Because the high affinity CaM binding IQ motif, which has been shown essential for CDF (Zühlke et al., 1999; Zühlke et al., 2000), resides within the C terminus of this peptide, further attention was focused on the peptide's N terminus as the potential CaMKII interaction site. One stretch of six residues within the 1639-1660 peptide, TVGKF(Y/I)A, was identified as being nearly identical in α_{1C} (Caᵥ1.2) and α_{1A} (Caᵥ2.1), the pore-forming subunit of P/Q-type Ca²⁺ channels, which display their own form of CDF (DeMaria et al., 2001; Lee et al., 1999). Accordingly, an α_{1C} fusion protein was constructed containing the amino acids EEDAAA in place of TVGKFY within an otherwise wild-type sequence of residues 1581-1690 (Mut6). CaMKII binding to the Mut6 fusion protein was reduced by 87.3±4.5% relative to binding to wild-type 1581-1690 fusion protein (FIG. 5D). In contrast, the same amino acid substitution left CaM binding to this mutant fusion protein unaffected (FIG. 5C).

Disruption of CaMKII Binding to the C-Terminus of α_{1C} Prevents CDF

Tests were run to determined whether this site was critical for CDF by introducing the Mut6 mutation into α_{1C} subunits of L-type channels expressed in Xenopus oocytes. Because L-type channels also display a strong Ca²⁺-dependent inactivation process (CDI) that could diminish the ability to detect facilitation, conditions were sought under which CDF could be observed uncontaminated by CDI. Fortunately, previous work has shown that even in heterologous systems, robust CDF during trains of depolarizing pulses can be obtained by a point mutation within the “IQ” motif (Zühlke et al., 1999; Zühlke et al., 2000). Removal of the bulky side-chain of the IQ isoleucine (I1654A) uncouples the Ca²⁺-sensor from the down-stream inactivation machinery (Kim et al., 2004), thereby providing a convenient platform for studying CDF in the absence of CDI. In this setting, the Mut6 modification of the CaMKII interaction site completely abolished CDF (FIG. 6). There was no potentiation of I_{Ca} at any point during the train of 40 successive depolarizations within the entire range of frequencies tested (0.5-3.3 Hz). Abolition of the Ca²⁺-dependent facilitatory process was also observed in experiments using a two-pulse protocol and finely graded changes in interpulse interval. Ca²⁺ currents evoked by the second pulse averaged 110% of those elicited by the first pulse at a time interval when the peak Ba²⁺ current had only recovered to ˜95% (FIG. 6D). A comparable difference between recovery of Ca²⁺ and Ba²⁺ currents was seen in wild-type α_{1C} (Zühlke et al., 1999; Zühlke et al., 2000), but was likewise abolished by the Mut6 modification (data not shown). Thus, in both of the approaches used to assess facilitation, potentiation of I_{Ca} during trains of depolarizations, and recovery from the after-effects of a single pulse, Ca²⁺ dependent facilitation was abolished by mutation of the CaMKII interaction site on Caᵥ1.2. These data support the hypothesis that CDF depends critically on the CaMKII interaction with α_{1C} at this C-terminal site.

The CaMKII Binding Site for the C-Terminus of α_{1C} is Conserved Among Multiple CaMKII Isoforms and Localizes to the Catalytic Domain

In addition to α-CaMKII, the predominantly brain-enriched isoform studied in the preceding experiments, there are several other CaMKII isoforms that differ in their cellular and subcellular distributions (Hudmon and Schulman, 2002a). For example, the major CaMKII isoforms are α and β in brain and δ in heart (Edman and Schulman, 1994). Of particular interest was the δ isoform, the major CaMKII isoform in the heart (Edman and Schulman, 1994). Accordingly, the generality of CaMKII interactions with the C-terminal tail of α_{1C} across a range of isoforms was examined. The α, β, γ_{B}, δ_{B}, and δ_{C} isoforms were transiently expressed in HEK293 cells for use as source material in pull-down assays and detected by the sensitive calmodulin overlay technique (Glenney and Weber, 1983) (FIG. 7A). In the absence of autophosphorylation, no binding was ever observed for any of the isoforms tested (data not shown). However, once auto-thiophosphorylated, robust binding to the α_{1C} C-terminal tail was observed for each of these CaMKII isoforms, with the sole exception of γ_{B}-CaMKII. Thus, the capability of interaction with Caᵥ1.2 is a widespread property of the CaMKII family, including the α/β and δ-isoforms prevalent in brain and cardiac tissue.

Where is the binding site for α_{1C} on CaMKII? The conserved nature of the α_{1C} binding site between brain and cardiac CaMKII isoforms favored a binding site that is conserved among the different kinase isoforms. As a starting point, the conserved catalytic domain of α-CaMKII was considered, based on a recent report describing its interaction with the C-terminus of the NR2B subunit of the NMDA receptor (Bayer et al., 2001). Indeed, binding of the C-terminal tail of α_{1C} to autophosphorylated CaMKII was blocked by a peptide modeled after the CaMKII binding site of the NR2B subunit (NR2B peptide, FIG. 7B). Further, binding of α_{1C} to CaMKII is potently blocked by peptides designed around Thr²⁸⁶ and the autoregulatory domain of CaMKII, including the peptide substrate AC-2 and the peptide inhibitor AC-3i (FIG. 7B) as well as AIP-2 (FIG. 2C). As expected, the control peptide AC-3c had no effect on binding. Both sets of observations resemble previous findings using peptide inhibition to study binding of CaMKII to NR2B (Bayer et al., 2001; Strack et al., 2000). A logical conclusion is that similar or identical molecular determinants on CaMKII are responsible for binding either to α_{1C} or to NR2B. The NR2B sequence that was found to support interaction with CaMKII closely resembles the autoregulatory domain of CaMKII surrounding Thr²⁸⁶ (Bayer et al., 2001) (FIG. 7C). In turn, both of these stretches of amino acids show significant resemblance to the region of α_{1C} that was identified as critical for CaMKII interaction by peptide competition (FIG. 5B), and that includes the TVGKFY sequence that was altered to the detriment of the α_{1C}-CaMKII interaction. Although the corresponding regions of α_{1C} and NR2B display points of sequence similarity (highlighted in FIG. 7C by dots and dashes), the overall degree of homology is limited.

CaMKII Binding to the C-Terminus of α_{1C} Produces a Dedicated Ca²⁺ Sensor

The functional nature of the channel-kinase interaction could follow one of a number of possible scenarios. During recurrent rises and falls in Ca²⁺, the enzyme might cycle on and off the channel. Alternatively, CaMKII might remain anchored to α_{1C} with its activity persistently switched on, like CaMKII associated with the NMDA receptor (Bayer et al., 2001). Finally, CaMKII might stay tethered to the α_{1C} subunit, like PKA associated with Caᵥ1.2 through an AKAP, but with kinase activity modulated by local changes in Ca²⁺/CaM, similar to the way that PKA is regulated by cAMP for β-adrenergic modulation (Gao et al., 1997). To explore these possibilities, tests were run to determine whether whether CaMKII dissociated from the C-terminal tail upon reversal of the Ca²⁺ elevation or the kinase activation that initially drove the interaction.

When the Ca²⁺ chelator EGTA was added immediately after the pre-autophosphorylation reaction, the binding of CaMKII to the α_{1C} C-terminal tail was inhibited (FIG. 8A). In contrast, after autophosphorylated CaMKII had bound to the α_{1C} C-terminal tail, EGTA in the wash buffer (two or three rounds of washing, each lasting ˜5 min) failed to dissociate the kinase (FIG. 8A). Dephosphorylation of autophosphorylated CaMKII with protein phosphatase 1 (PP1) before presenting the kinase to the α_{1C} C-terminal fusion protein prevented binding (FIG. 8B). However, dephosphorylation of CaMKII after binding did not. Even the combination of dephosphorylation and EGTA application failed to reverse binding (FIG. 8B). In control experiments, immunoblotting with the phospho-specific antibody indicated that Thr²⁸⁶ had been completely dephosphorylated by PP1 treatment after the initial kinase binding (FIG. 8B). Thus, while Ca²⁺/CaM and autophosphorylation were necessary for CaMKII to bind to the α_{1C} C-terminus, the same conditions were no longer required to sustain the interaction.

Tethered CaMKII Retains its Dependence on Ca²⁺/CaM for Activity

Since the CaMKII binding for α_{1C} and for NR2B both appear to localize to the catalytic domain of the kinase, the question was posed as to whether α_{1C} binding to CaMKII regulates its kinase activity, as in the case of NR2B. When bound to NR2B, CaMKII remains active in phosphorylating substrates even in the absence of Ca²⁺/CaM and autophosphorylation (Bayer et al., 2001). To determine how CaMKII is regulated when it is stably bound to the α_{1C} C-terminus, the Ca²⁺/CaM-dependent was compared to the Ca²⁺/CaM-independent (autonomous) activity following PP1 treatment. Dephosphorylation by PP1, assessed by tracking the loss of autonomous activity for soluble kinase, was complete within 30 min (FIG. 8C). Under similar conditions, it was observed that treatment of α_{1C}-bound kinase with PP1 completely eliminated autonomous activity (remaining activity was 1.2±0.6% of that without PP1 treatment) (FIG. 8D). Thus, autonomous activity of bound CaMKII was not maintained merely by interaction of the kinase with the α_{1C} C-terminus, but depended strictly upon CaMKII autophosphorylation.

Following PP1 treatment, tethered CaMKII could be re-activated by Ca²⁺/CaM. In these respects, CaMKII binding to α_{1C} or to NR2B had very different effects on the activity of the kinase. As discussed below, the association of CaMKII to the α_{1C} C-terminus is well-suited to localize the kinase in close proximity to its regulatory target, but not to keep the kinase constitutively active.

Discussion

Ca²⁺-dependent facilitation is a powerful positive feedback mechanism that allows excitable cells such as neurons and myocytes to modulate Ca²⁺ entry through Ca²⁺ channels according to the previous pattern of repetitive activity. The functional consequences are clearest in heart, where CDF of L-type channels is required for sinoatrial pacemaker activity (Vinogradova et al., 2000), and contributes to the myocardial force-frequency relationship, a form of adaptive plasticity that has intrigued investigators ever since Bowditch's work in the late 1800s (Koch-Weser and Blinks, 1963). However, CDF or related phenomena have also been described for voltage-gated Ca²⁺ channels in neurons (Cuttle et al., 1998), smooth muscle cells (McCarron et al., 1992) and adrenal glomerulosa cells (Wolfe et al., 2002). Although not described in neurons, CDF of L-type channels could play a major role in supporting their privileged status in mediating excitation-transcription coupling and long-term synaptic plasticity (Bradley and Finkbeiner, 2002; Deisseroth et al., 2003; West et al., 2002).

Several new findings are presented that advance the understanding of CDF at the cellular or molecular level: first, L-type currents in neurons are capable of CDF, with the same Ca²⁺- and CaMKII-dependence as previously found in cardiac myocytes; second, CaMKII associates with the pore-forming α_{1C} subunit of L-type channels in brain homogenates as evidenced by the co-immunoprecipitation studies; third, specific regions of α_{1C} subunit have the capability of directly anchoring activated CaMKII; fourth, CaMKII can phosphorylate α_{1C} in regions of previously implicated in regulating channel function; fifth, a mutation in the C-terminus of α_{1C} that disrupts CaMKII binding to that region completely abolished CDF; and sixth, once tethered to the C-terminus, CaMKII can be completely dephosphorylated and deactivated, yet persist in its association and retains its dependence on Ca²⁺/CaM. Thus, it is concluded that the targeting and localization of CaMKII to its substrate is critical to its known regulatory function, the first instance of such a relationship. It is also concluded that the targeting and localization of CaMKII to the L-type channel is critical for CDF. These experiments suggest that individual L-type channels can take advantage of CaMKII as a frequency detector for the regulation of their Ca²⁺ influx. The tethered kinase provides a local and specific integrator of preceding channel activity that controls future channel function through feed-forward autoregulation.

A Working Model for Unifying Disparate Observations on CDF

The findings provide a biochemical and molecular explanation of the earlier electrophysiological findings that first suggested that CDF was mediated by CaMKII. Ca²⁺ buffer experiments revealed that CDF depended on a calcium signal near the channel (Hryshko and Bers, 1990). Pharmacological inhibition of CaMKII abolished CDF (Anderson et al., 1994; Xiao et al., 1994; Yuan and Bers, 1994). Immunostaining showed that autophosphorylated CaMKII was concentrated near the surface membrane of cardiomyocytes (Xiao et al., 1994). More recently, Anderson and colleagues found that direct application of thiophosphorylated (constitutively-activated) CaMKII to the cytoplasmic face of cardiac myocyte membranes induced a high Pₒₚₑₙ mode of L-type channel activity, thereby accounting for CDF; the modulatory effect could be prevented by non-hydrolyzable ATP analogs or CaMK blockers, further implicating CaM kinase activity (Dzhura et al., 2000).

The results in this paper not only uncover key molecular underpinnings of those earlier studies, but also resolve several unanswered questions. How can a ubiquitous CaMKII fulfill the requirement for a local Ca²⁺ signal in CDF (Hryshko and Bers, 1990; Vinogradova et al., 2000)? How can one reconcile the classical conception of CaMKII as a widely diffusible, multifunctional regulator of overall cellular activity with the finding that a local Ca²⁺ signal is essential for CDF (Hryshko and Bers, 1990; Vinogradova et al., 2000)? Is autophosphorylated CaMKII concentrated near the cell surface (Vinogradova et al., 2000; Xiao et al., 1994) simply because Ca²⁺ is highest near sites of influx (Hryshko and Bers, 1990)? Is a membrane localization of CaMKII achieved by tethering to L-type channels and is such targeting necessary for CDF? Does CaMKII mediate CDF by directly phosphorylating the pore-forming α_{1C} subunit or an auxiliary protein (Anderson et al., 1994)?

Answers to these questions can be put forward in the context of a working model of the L-type channel-CaMKII interaction during CDF (FIG. 8E). The experimental results lead to the following hypothesis. In an excitable cell that had been quiescent for a long time, CaMKII is free in the cytoplasm (lower left) inasmuch as the inactive form of the kinase did not significantly interact with any of the cytoplasmic regions of α_{1C}. After an initial Ca²⁺ entry, recruitment to the channel takes place in an activity-dependent manner. CaM binding to soluble CaMKII targets the kinase to certain intracellular domains of α_{1C}, and if the depolarization frequency suffices to produce CaMKII autophosphorylation on Thr²⁸⁶, the resulting displacement of the kinase's autoregulatory domain exposes a potent anchoring site for the α_{1C} C-terminus (lower middle). Observations that autophosphorylated CaMKII is concentrated at the myocyte sarcolemma (Vinogradova et al., 2000; Xiao et al., 1994), can be explained at least in part by a direct interaction of the kinase with α_{1C}. Moreover the requirement for a local Ca²⁺ signal to trigger CDF (Hryshko and Bers, 1990; Vinogradova et al., 2000) would arise if the necessary phosphorylation could only be achieved by a tethered kinase that is modulated by CaM molecules in the immediate vicinity of the channel-anchored CaMKII.

Once established, this interaction may persist for many minutes even after Ca²⁺ is lowered and the kinase is completely dephosphorylated (FIG. 8D), so that CaMKII remains tightly tethered to the channel so long as the cell is intermittently active (lower right). This scenario capitalizes on the dodecameric structure of the CaMKII holoenzyme (Kolodziej et al., 2000) by using one or more kinase subunits for the purpose of subcellular localization (upper left). The securing of CaMKII in close proximity to key substrate site(s) on intracellular loops of the channel protein produces a high rate of channel phosphorylation and promotes a pattern of gating with high Pₒ (mode 2). (Dzhura et al., 2000) Lowering of the frequency of Ca²⁺ influx reduces kinase activation and allows phosphatases to prevail in dephosphorylating both the channel and its associated CaMKII, driving the channel into a low Pₒ gating mode (upper right). Because the resident CaMKII can be fully dephosphorylated while remaining associated with the channel, its modulatory activity can be graded over the widest possible working range.

These observations provide a molecular explanation of key findings of the earlier studies. Autophosphorylated CaMKII is concentrated at the myocyte sarcolemma (Vinogradova et al., 2000; Xiao et al., 1994), at least in part because of a direct interaction with α_{1C}. CDF requires a local Ca²⁺ signal, one that can be suppressed by the fast Ca²⁺ buffer BAPTA but not the slow Ca²⁺ buffer EGTA (Hryshko and Bers, 1990; Vinogradova et al., 2000), because the targets of the Ca²⁺ signaling are CaM molecules in the immediate vicinity of the channel-anchored CaMKII. By virtue of its position, the anchored kinase has a tremendous kinetic advantage over cytosolic CaMKII molecules and essentially monopolizes the modulatory function. Accordingly, a mutation in α_{1C} that rendered the cytoplasmic tail unable to bind CaMKII completely abolished CDF (FIG. 6). Together, the channel-kinase complex represents a dedicated frequency detector that responds specifically to local Ca²⁺ signaling.

Looking beyond Ca²⁺ channels in surface membranes, Ca²⁺ sequestration into intracellular Ca²⁺ stores undergoes a frequency-dependent acceleration in myocardial cells, also critically dependent on CaMKII (DeSantiago et al., 2002). It remains unclear whether this action of CaMKII depends on activity-dependent targeting, and whether frequency-dependent modulation is a common feature of Ca²⁺ signaling proteins (Maier and Bers, 2002).

Comparisons with L-Type Channel Modulation by Other Kinases: PKA Localization and Action

The tethering of CaMKII to α_{1C} provides a fresh example of a system in which effector proteins such as kinases and phosphatases are linked to substrates. And adds some unique elements to the repertoire of mechanisms used by signaling molecules to link stimulus to cellular response. The L-type channel-CaMKII interaction takes advantage of the multimeric CaMKII holoenzyme, utilizing one or a limited number of its 12 catalytic subunits for anchoring and therefore circumventing the use of auxiliary proteins such as AKAPs or RACKs, which tether PKA or PKC, respectively (Bunemann et al., 1999; Dorn and Mochly-Rosen, 2002; Schechtman and Mochly-Rosen, 2001; Tavalin et al., 1999). It is particularly interesting to compare these findings on the anchoring of CaMKII with the linking of protein kinase A to its substrates by AKAPs (Tavalin et al., 1999). There are similarities in the tuning of the kinetics and specificity of functional effects, but significant differences in the mechanism of targeting. AKAPs (and RACKs, their functional equivalents for PKC) are modular adapter proteins that physically link the kinase to its substrate (Bunemann et al., 1999; Dorn and Mochly-Rosen, 2002; Schechtman and Mochly-Rosen, 2001; Tavalin et al., 1999). In contrast, no such auxiliary proteins have yet been uncovered for localization of CaMKII; the use of individual subunits for anchoring the holoenzyme may minimize the need for intermediary modules. Another distinction lies in the persistent tethering of CaMKII and its catalytic domains to α_{1C} and in the nature of the entity that is anchored. AKAPs interact with the regulatory subunit of PKA, not the catalytic subunit, so dissociation of the R₂C₂ complex leads to the immediate loss of catalytic localization once the C subunits are liberated and thereby activated. In muscle cells, PKA and AKAPs form part of a signaling complex for β-adrenergic mediated potentiation of L-type Ca²⁺ currents (Gao et al., 1997; Hulme et al., 2002). Perhaps the critical role of AKAPs is restricted to a one-time localization of the catalytic subunits, acceptable if the β-adrenergic signaling requires rapid responsiveness but only on infrequent occasions. The spatial zone of catalytic activity is delimited by the distance from site of anchored subunit to most distant subunit of that holoenzyme. Dissociation of the PKA R₂C₂ complex from AKAPs leads to the immediate loss of catalytic localization once the C subunits are liberated and thereby activated over a much larger spatial volume. This mechanism is ideal for enabling catalytic subunits to diffuse from site of activation to the nucleus and is acceptable if β-adrenergic potentiation of L-type Ca²⁺ currents (Gao et al., 1997; Hulme et al., 2002) requires rapid responsiveness but only on infrequent occasions. The persistent tethering of the entire CaMKII holoenzyme, accomplished through the tethering of an integral subunit, might be much better suited for continuous operation as an integrator of the previous history of excitation and of L-type Ca²⁺ channel activity, endowed with a rapid on-off rate and dedicated to a limited number of channels.

Finally, it is speculated that the multimeric nature of CaMKII and the multiplicity of potential binding interactions on various cytoplasmic domains of α_{1C} could create a large number of distinct binding states. A specific binding state could reflect the preceding pattern of intracellular Ca²⁺ signaling.

This is the first evidence that CaMKII directly phosphorylates the α_{1C} subunit. A major effort will be needed to narrow down the phosphorylation sites to specific residues and to evaluate their relative contributions to the modulatory action on channel activity. Among the several cytoplasmic loops that have been implicated in Ca²⁺-dependent gating (Adams and Tanabe, 1997), the C-terminus is a reasonable starting point, since this region appears functionally important for L-type channel potentiation by PKA or c-Src (Bence-Hanulec et al., 2000; Rotman et al., 1995). However, the putative target for PKA, Ser¹⁹²⁸, is unlikely to be key to CaMKII modulation: the surrounding amino acids deviate from consensus sites for CaMKII (White et al., 1998), and PKA-mediated potentiation leads to enhancement of both mode 1 and mode 2 gating (Yue et al., 1990), whereas CaMKII-mediated facilitation leads to enhancement of mode 2 only (Dzhura et al., 2000). Phosphorylation of the N-terminus by CaMKII would also be interesting to pursue, given that removal of the α_{1C} N-terminus enhances channel current, as if it formed part of an inhibitory gate (Ivanina et al., 2000). Kinase-substrate interactions cannot be the sole explanation of CaMKII anchoring, since binding to the GST-fusion proteins encoding other intracellular loops was observed in the absence of phosphorylation, and the regions for binding and phosphorylation do not coincide. While the focus has been on CaMKII interactions with the pore-forming α_{1C} subunit, additional binding to or phosphorylation of auxiliary Ca²⁺ channel subunits cannot be excluded.

Similarities and Contrasts with CaMKII-NMDAR Interactions

Like L-type channels, NMDA receptors are predominant Ca²⁺ entry pathways in neurons for triggering synaptic plasticity and signaling to the nucleus and CaMKII is tethered to the NR1 and NR2B subunits of the NMDA receptor, so these experiments provide interesting points of comparison with previous work showing the direct binding of CaMKII to the NR2B and NR1 subunits of NMDARs (Bayer et al., 2001; Leonard et al., 2002; Leonard et al., 1999; Strack and Colbran, 1998; Strack et al., 2000). There are telling similarities between NMDAR subunits and α_{1C} as targets for CaMKII binding. First, completely inactive CaMKII will not initiate binding to any of these subunits. Second, in both NR2B and ═_{1C}, a C-terminal domain of the membrane protein competes with the autoregulatory domain of CaMKII for binding to the kinase, as shown by peptide competition (Strack et al., 2000) (FIG. 7B). This similarity was driven home by the finding that a peptide based on the CaMKII binding site on NR2B prevented the kinase from interacting with the α_{1C} C-terminal tail (FIG. 7B). Third, in both NR1 and α_{1C}, the site of CaMKII binding lies close to a site for CaM binding. In the C0 domain of NR1, the amino acids most critical for CaMKII binding lie three residues N-terminal to those most important for CaM binding (Leonard et al., 2002). Likewise, the α_{1C} sequence implicated in the CaMKII interaction (Mut6) lies between stretches of amino acids, among them the IQ motif, that are critical for CaM tethering and effector action (Pate et al., 2000; Peterson et al., 1999; Pitt et al., 2001; Romanin et al., 2000; Zühlke et al., 1999; Zühlke et al., 2000). Further studies will be needed to understand how the activity of the anchored CaMKII may be integrated with the Ca²⁺-sensing properties of the CaM-IQ domain complex for regulation of L-type channel gating and for downstream signaling to nuclear CREB (Dolmetsch et al., 2001).

There are also critical functional differences between α_{1C} and NR2B in their interaction with CaMKII. Although the C-terminal tails of α_{1C} and NR2B use a biochemically similar, if not identical site on CaMKII for binding, the different anchoring proteins produce significant differences in how this binding site is exposed and how kinase activity is affected. Although the C-terminal tails of α_{1C} and NR2B use overlapping sites on CaMKII for binding, the two channels exhibit significant differences in kinase activation state requirements and in consequences of tethering. The NR2B C-terminus displays a high affinity interaction with CaMKII that merely requires Ca²⁺/CaM activation of CaMKII, not autophosphorylation (Bayer et al., 2001). In contrast, the C-terminus of α_{1C} only binds to autophosphorylated CaMKII (FIG. 4). Binding of CaMKII to NR2B alters kinase function, causing maintained kinase activity even in the absence of Ca²⁺/CaM or autophosphorylation. This is not the case for CaMKII binding to α_{1C}; these experiments show that interaction with the α_{1C} C-terminus does not circumvent the autoinhibitory function of the bound kinase. The contrasting properties might arise from substantial differences in the respective C-terminal sequences of α_{1C} and NR2B (FIG. 7C) and might offer specific advantages appropriate to the different roles of the two channels. The establishment of sustained CaMKII activity following transient NMDA receptor signaling seems perfectly appropriate as a means of supporting enduring effects—long-term potentiation and long-term depression, for example (Lisman et al., 2002).

Recent work suggests that CaMKII interaction with the Drosophila eag potassium channel recapitulates the NMDAR type of regulation, as the binding of CaMKII to the C-terminus of Eag produces a form of constitutive activity that is uncoupled from the Ca²⁺ signal (Sun et al., 2004). As in the case with NMDAR signaling, constitutive kinase activity is an appropriate signaling mode for encoding the persistent responses underlying synaptic plasticity. On the other hand, Ca²⁺-dependent facilitation would suffer a significant loss of dynamic range if the α_{1C} C-terminal interaction with CaMKII were to cause constitutive kinase activity. The retention of dependence on Ca²⁺/CaM for enzymatic activity is well-suited for the operation of CaMKII as a built-in integrator of the frequency of prior Ca²⁺ signaling (Hudmon and Schulman, 2002a; Maier and Bers, 2002).

### REFERENCES

U.S. Pat. No. 6,518,245, Feb. 11, 2003 (Anderson et al.).
Adams, B., and Tanabe, T. (1997). Structural Regions of the Cardiac Ca Channel α_{1C} Subunit Involved in Ca-dependent Inactivation. J Gen Physiol 110, 379-389.
Anderson, M., Braun, A., Schulman, H., and Premack, B. (1994). Multifunctional Ca²⁺/calmodulin-dependent protein kinase mediates Ca²⁺-induced enhancement of the L-type Ca²⁺ current in rabbit ventricular myocytes. Circ Res 75, 854-861.
Bayer, K. U., De Koninck, P., Leonard, A. S., Hell, J. W., and Schulman, H. (2001). Interaction with the NMDA receptor locks CaMKII in an active conformation. Nature 411, 801-805.
Bayer, K. U., De Koninck, P., and Schulman, H. (2002). Alternative splicing modulates the frequency-dependent response of CaMKII to Ca²⁺ oscillations. Embo J 21, 3590-3597.
Bence-Hanulec, K. K., Marshall, J., and Blair, L. A. (2000). Potentiation of Neuronal L Calcium Channels by IGF-1 Requires Phosphorylation of the α₁ Subunit on a Specific Tyrosine Residue. Neuron 27, 121-131.
Borst, J. G., and Sakmann, B. (1998). Facilitation of presynaptic calcium currents in the rat brainstem. J Physiol 513, 149-155.
Bradley, J., and S. Finkbeiner. 2002. An evaluation of specificity in activity-dependent gene expression in neurons. Prog Neurobiol. 67:469-77.
Bradshaw, J. M., Hudmon, A., and Schulman, H. (2002). Chemical quenched flow kinetic studies indicate an intraholoenzyme autophosphorylation mechanism for Ca²⁺/calmodulin-dependent protein kinase II. J Biol Chem 277, 20991-20998.
Bradshaw, J. M., Kubota, Y., Meyer, T., and Schulman, H. (2003). An ultrasensitive Ca²⁺/calmodulin-dependent protein kinase II-protein phosphatase 1 switch facilitates specificity in postsynaptic calcium signaling. Proc Natl Acad Sci U S A 100, 10512-10517.
Braun, A. P., and Schulman, H. (1995). A non-selective cation current activated via the multifunctional Ca²⁺-calmodulin-dependent protein kinase in human epithelial cells. J Physiol 488 (Pt 1), 37-55.
Bunemann, M., Gerhardstein, B. L., Gao, T., and Hosey, M. M. (1999). Functional Regulation of L-type Calcium Channels via Protein Kinase A-mediated Phosphorylation of the β₂ Subunit. J Biol Chem 274, 33851-33854.
Cai, D., Mulle, J. G., and Yue, D. T. (1997). Inhibition of recombinant Ca²⁺ channels by benzothiazepines and phenylalkylamines: class-specific pharmacology and underlying molecular determinants. Mol Pharmacol 51, 872-881.
Cuttle, M. F., Tsujimoto, T., Forsythe, I. D., and Takahashi, T. (1998). Facilitation of the presynaptic calcium current at an auditory synapse in rat brainstem. J Physiol 512, 723-729.
De Jongh, K. S., B. J. Murphy, A. A. Colvin, J. W. Hell, M. Takahashi, and W. A. Catterall. 1996. Specific Phosphorylation of a Site in the Full-Length Form of the al Subunit of the Cardiac L-Type Calcium Channel by Adenosine 3′,5′-Cyclic Monophosphate-Dependent Protein Kinase. Biochemistry. 35:10392-402.
De Koninck, P., and Schulman, H. (1998). Sensitivity of CaM Kinase II to the Frequency of Ca²⁺ Oscillations. Science 279, 227-230.
Deisseroth, K., P. G. Mermelstein, H. Xia, and R. W. Tsien. 2003. Signaling from synapse to nucleus: the logic behind the mechanisms. Curr Opin Neurobiol. 13:354-65.
DeMaria, C. D., Soong, T. W., Alseikhan, B. A., Alvania, R. S., and Yue, D. T. (2001). Calmodulin bifurcates the local Ca²⁺ signal that modulates P/Q-type Ca²⁺ channels. Nature 411, 484-489.
DeSantiago, J., Maier, L. S., and Bers, D. M. (2002). Frequency-dependent acceleration of relaxation in the heart depends on CaMKII, but not phospholamban. J Mol Cell Cardiol 34, 975-984.
Dolmetsch, R. E., Pajvani, U., Fife, K., Spotts, J. M., and Greenberg, M. E. (2001). Signaling to the Nucleus by an L-type Calcium Channel-Calmodulin Complex Through the MAP Kinase Pathway. Science 294, 333-339.
Dorn, G. W., 2nd, and Mochly-Rosen, D. (2002). Intracellular transport mechanisms of signal transducers. Annu Rev Physiol 64, 407-429.
Dzhura, I., Wu, Y., Colbran, R. J., Balser, J. R., and Anderson, M. E. (2000). Calmodulin kinase determines calcium-dependent facilitation of L-type calcium channels. Nat Cell Biol 2, 173-177.
Edman, C. F., and Schulman, H. (1994). Identification and characterization of δ_{B}-CaM kinase and δ_{C}-CaM kinase from rat heart, two new multifunctional Ca²⁺/calmodulin-dependent protein kinase isoforms. Biochim Biophys Acta 1221, 89-101.
Erickson, M. G., H. Liang, M. X. Mori, and D. T. Yue. 2003. FRET Two-Hybrid Mapping Reveals Function and Location of L-Type Ca²⁺ Channel CaM Preassociation. Neuron. 39:97-107.
Eshete, F., and R. D. Fields. 2001. Spike frequency decoding and autonomous activation of Ca2+-calmodulin-dependent protein kinase II in dorsal root ganglion neurons. J Neurosci. 21:6694-705.
Feldman, M. D., Alderman, J. D., Aroesty, J. M., Royal, H. D., Ferguson, J. J., Owen, R. M., Grossman, W., and McKay, R. G. (1988). Depression of systolic and diastolic myocardial reserve during atrial pacing tachycardia in patients with dilated cardiomyopathy. J Clin Invest 82, 1661-1669.
Gao, T., Cuadra, A. E., Ma, H., Bunemann, M., Gerhardstein, B. L., Cheng, T., Eick, R. T., and Hosey, M. M. (2001). C-terminal Fragments of the α_{1C} (Caᵥ1.2) Subunit Associate with and Regulate L-type Calcium Channels Containing C-terminal-truncated α_{1C} Subunits. J Biol Chem 276, 21089-21097.
Gao, T., Yatani, A., Dell'Acqua, M. L., Sako, H., Green, S. A., Dascal, N., Scott, J. D., and Hosey, M. M. (1997). cAMP-Dependent Regulation of Cardiac L-type Ca²⁺ Channels Requires Membrane Targeting of PKA and Phosphorylation of Channel Subunits. Neuron 19, 185-196.
Glenney, J. R., Jr., and Weber, K. (1983). Detection of calmodulin-binding polypeptides separated in SDS-polyacrylamide gels by a sensitive [¹²⁵I]calmodulin gel overlay assay. Methods Enzymol 102, 204-210.
Hanson, P. I., Kapiloff, M. S., Lou, L. L., Rosenfeld, M. G., and Schulman, H. (1989). Expression of a multifunctional Ca²⁺/calmodulin-dependent protein kinase and mutational analysis of its autoregulation. Neuron 3, 59-70.
Hasenfuss, G., Holubarsch, C., Hermann, H. P., Astheimer, K., Pieske, B., and Just, H. (1994). Influence of the force-frequency relationship on haemodynamics and left ventricular function in patients with non-failing hearts and in patients with dilated cardiomyopathy. Eur Heart J 15, 164-170.
Hell, J. W., Yokoyama, C. T., Breeze, L. J., Chavkin, C., and Catterall, W. A. (1995). Phosphorylation of presynaptic and postsynaptic calcium channels by cAMP-dependent protein kinase in hippocampal neurons. EMBO J 14, 3036-3044.
Hryshko, L. V., and Bers, D. M. (1990). Ca current facilitation during postrest recovery depends on Ca entry. Am J Physiol 259, H951-961.
Hudmon, A., Aronowski, J., Kolb, S. J., and Waxham, M. N. (1996). Inactivation and self-association of Ca²⁺/calmodulin-dependent protein kinase II during autophosphorylation. J Biol Chem 271, 8800-8808.
Hudmon, A., and Schulman, H. (2002a). NEURONAL CA²⁺/CALMODULIN-DEPENDENT PROTEIN KINASE II: The Role of Structure and Autoregulation in Cellular Function. Annu Rev Biochem 71, 473-510.
Hudmon, A., and Schulman, H. (2002b). Structure/Function of the Multifunctional Ca²⁺/Calmodulin-Dependent Protein Kinase II. Biochem J 4.
Hulme, J. T., Ahn, M., Hauschka, S. D., Scheuer, T., and Catterall, W. A. (2002). A novel leucine zipper targets AKAP15 and cyclic AMP-dependent protein kinase to the C terminus of the skeletal muscle Ca2+ channel and modulates its function. J Biol Chem 277, 4079-4087.
Ikeda, A., S. Okuno, and H. Fujisawa. 1991. Studies on the generation of Ca²⁺/calmodulin-independent activity of calmodulin-dependent protein kinase II by autophosphorylation. Autothiophosphorylation of the enzyme. J Biol Chem. 266:11582-8.
Ivanina, T., Blumenstein, Y., Shistik, E., Barzilai, R., and Dascal, N. (2000). Modulation of L-type Ca²⁺ Channels by Gβγ and Calmodulin via Interactions with N and C Termini of α_{1C}. J Biol Chem 275, 39846-39854.
Kim, J., Ghosh, S., Nunziato, D. A., and Pitt, G. S. (2004). Isolation of the Components Controlling Inactivation of Voltage-Gated Ca²⁺ channels. Neuron 41, 745-754.
Koch-Weser, J., and Blinks, J. (1963). The influence of the interval between beats on myocardial contractility. Pharmacology Review 15, 601-652.
Kolodziej, S. J., Hudmon, A., Waxham, M. N., and Stoops, J. K. (2000). Three-dimensional reconstructions of calcium/calmodulin-dependent (CaM) kinase IIα and truncated CaM kinase IIα reveal a unique organization for its structural core and functional domains. J Biol Chem 275, 14354-14359.
Lai, Y., A. C. Nairn, F. Gorelick, and P. Greengard. 1987. Ca²⁺/calmodulin-dependent protein kinase II: identification of autophosphorylation sites responsible for generation of Ca²⁺/calmodulin-independence. Proc Natl Acad Sci U S A. 84:5710-4.
Lee, A., Wong, S. T., Gallagher, D., Li, B., Storm, D. R., Scheuer, T., and Catterall, W. A. (1999). Ca2+/calmodulin binds to and modulates P/Q-type calcium channels. Nature 399, 155-159.
Lee, K. S. (1987). Potentiation of the calcium-channel currents of internally perfused mammalian heart cells by repetitive depolarization. Proc Natl Acad Sci U S A 84, 3941-3945.
Leonard, A. S., Bayer, K.-U., Merrill, M. A., Lim, I. A., Shea, M. A., Schulman, H., and Hell, J. W. (2002). Regulation of Calcium/Calmodulin-dependent Protein Kinase II Docking to N-Methyl-D-aspartate Receptors by Calcium/Calmodulin and α-Actinin. J Biol Chem 277, 48441-48448.
Leonard, A. S., Lim, I. A., Hemsworth, D. E., Horne, M. C., and Hell, J. W. (1999). Calcium/calmodulin-dependent protein kinase II is associated with the N-methyl-D-aspartate receptor. Proc Natl Acad Sci U S A 96, 3239-3244.
Lisman, J., Schulman, H., and Cline, H. (2002). THE MOLECULAR BASIS OF CaMKII FUNCTION IN SYNAPTIC AND BEHAVIOURAL MEMORY. Nat Rev Neurosci 3, 175-190.
Lou, L. L., and H. Schulman. 1989. Distinct autophosphorylation sites sequentially produce autonomy and inhibition of the multifunctional Ca2+/calmodulin-dependent protein kinase. J Neurosci. 9:2020-32.
Lou, L. L., Lloyd, S. J., and Schulman, H. (1986). Activation of the multifunctional Ca²⁺/calmodulin-dependent protein kinase by autophosphorylation: ATP modulates production of an autonomous enzyme. Proc Natl Acad Sci U S A 83, 9497-9501.
Maier, L. S., and Bers, D. M. (2002). Calcium, calmodulin, and calcium-calmodulin kinase II: heartbeat to heartbeat and beyond. J Mol Cell Cardiol 34, 919-939.
Marban, E., and Tsien, R. W. (1982). Enhancement of calcium current during digitalis inotropy in mammalian heart: positive feed-back regulation by intracellular calcium? J Physiol 329, 589-614.
Mattson, M. (2004). Pathway towards and away from Alzheimer's disease. Nature 430, 631-639.
McCarron, J. G., McGeown, J. G., Reardon, S., Ikebe, M., Fay, F. S., and Walsh, J. V., Jr. (1992). Calcium-dependent enhancement of calcium current in smooth muscle by calmodulin-dependent protein kinase II. Nature 357, 74-77.
McHugh, D., Sharp, E. M., Scheuer, T., and Catterall, W. A. (2000). Inhibition of cardiac L-type calcium channels by protein kinase C phosphorylation of two sites in the N-terminal domain. PNAS 97, 12334-12338.
Meyer, T., Hanson, P. I., Stryer, L., and Schulman, H. (1992). Calmodulin trapping by calcium-calmodulin-dependent protein kinase. Science 256, 1199-1202.
Miller, S. G., Patton, B. L., and Kennedy, M. B. (1988). Sequences of autophosphorylation sites in neuronal type II CaM kinase that control Ca²⁺-independent activity. Neuron 1, 593-604.
Mulieri, L. A., Hasenfuss, G., Leavitt, B., Allen, P. D., and Alpert, N. R. (1992). Altered myocardial force-frequency relation in human heart failure. Circulation 85, 1743-1750.
Noble, S., and Shimoni, Y. (1981). The calcium and frequency dependence of the slow inward current ‘staircase’ in frog atrium. J Physiol 310, 57-75.
Pate, P., Mochca-Morales, J., Wu, Y., Zhang, J. Z., Rodney, G. G., Serysheva, II, Williams, B. Y., Anderson, M. E., and Hamilton, S. L. (2000). Determinants for Calmodulin Binding on Voltage-dependent Ca²⁺ channels. J Biol Chem 275, 39786-39792.
Peterson, B. Z., DeMaria, C. D., Adelman, J. P., and Yue, D. T. (1999). Calmodulin is the Ca²⁺ Sensor for Ca²⁺-Dependent Inactivation of L-type Calcium Channels [published erratum appears in Neuron 1999 April; 22(4):following 893]. Neuron 22, 549-558.
Piedras-Renteria, E. S., and Tsien, R. W. (1998). Antisense oligonucleotides against α_{1E} reduce R-type calcium currents in cerebellar granule cells. Proc Natl Acad Sci U S A 95, 7760-7765.
Pitt, G. S., Zuhlke, R. D., Hudmon, A., Schulman, H., Reuter, H., and Tsien, R. W. (2001). Molecular Basis of Calmodulin Tethering and Ca²⁺-dependent Inactivation of L-type Ca²⁺ channels. J Biol Chem 276, 30794-30802.
Randall, A., and Tsien, R. W. (1995). Pharmacological dissection of multiple types of Ca²⁺ channel currents in rat cerebellar granule neurons. J Neurosci 15, 2995-3012.
Romanin, C., Gamsjaeger, R., Kahr, H., Schaufler, D., Carlson, O., Abernethy, D. R., and Soldatov, N. M. (2000). Ca²⁺ sensors of L-type Ca²⁺ channel. FEBS Lett 487, 301-306.
Ross, J., Jr, Miura, T., Kambayashi, M., Eising, G. P., and Ryu, K.-H. (1995). Adrenergic Control of the Force-Frequency Relation. Circulation 92, 2327-2332.
Rotman, E. I., Murphy, B. J., and Catterall, W. A. (1995). Sites of selective cAMP-dependent phosphorylation of the L-type calcium channel alpha 1 subunit from intact rabbit skeletal muscle myotubes. J Biol Chem 270, 16371-16377.
Schechtman, D., and Mochly-Rosen, D. (2001). Adaptor proteins in protein kinase C-mediated signal transduction. Oncogene 20, 6339-6347.
Schouten, V. J., and Morad, M. (1989). Regulation of Ca²⁺ current in frog ventricular myocytes by the holding potential, c-AMP and frequency. Pflugers Arch 415, 1-11.
Singla, S. I., Hudmon, A., Goldberg, J. M., Smith, J. L., and Schulman, H. (2001). Molecular Characterization of Calmodulin Trapping by Calcium/Calmodulin-dependent Protein Kinase II. J Biol Chem 276, 29353-29360.
Strack, S., and Colbran, R. J. (1998). Autophosphorylation-dependent targeting of calcium/calmodulin-dependent protein kinase II by the NR2B subunit of the N-methyl- D-aspartate receptor. J Biol Chem 273, 20689-20692.
Strack, S., McNeill, R. B., and Colbran, R. J. (2000). Mechanism and Regulation of Calcium/Calmodulin-dependent Protein Kinase II Targeting to the NR2B subunit of the N-Methyl-D-aspartate Receptor. J Biol Chem 275, 23798-23806.
Sun, X. X., Hodge, J. J. L., Zhou, Y., Nguyen, M., and Griffith, L. C. (2004). The eag Potassium Channel Binds and Locally Activates Calcium/Calmodulin-dependent Protein Kinase II. J Biol Chem 279, 10206-10214.
Tavalin, S. J., Westphal, R. S., Colledge, M., Langeberg, L. K., and Scott, J. D. (1999). The molecular architecture of neuronal kinase/phosphatase signalling complexes. Biochem Soc Trans 27, 539-542.
Tymianski, A. (2003). Molecular mechanisms of calcium-dependent neurodegeneration in excitocity. Cell Calcium 34(4-5):325-37.
Vinogradova, T. M., Zhou, Y.-Y., Bogdanov, K. Y., Yang, D., Kuschel, M., Cheng, H., and Xiao, R.-P. (2000). Sinoatrial Node Pacemaker Activity Requires Ca²⁺/Calmodulin-Dependent Protein Kinase II Activation. Circ Res 87, 760-767.
West, A. E., E. C. Griffith, and M. E. Greenberg. 2002. Regulation of transcription factors by neuronal activity. Nat Rev Neurosci. 3:921-31.
White, R. R., Kwon, Y. G., Taing, M., Lawrence, D. S., and Edelman, A. M. (1998). Definition of Optimal Substrate Recognition Motifs of Ca²⁺-Calmodulin-dependent Protein Kinases IV and II Reveals Shared and Distinctive Features. J Biol Chem 273, 3166-3172.
Wolfe, J. T., Wang, H., Perez-Reyes, E., and Barrett, P. Q. (2002). Stimulation of recombinant Caᵥ3.2, T-type, Ca²⁺ channel currents by CaMKIIyc. J Physiol 538, 343-355.
Xiao, R. P., Cheng, H., Lederer, W. J., Suzuki, T., and Lakatta, E. G. (1994). Dual regulation of Ca²⁺/calmodulin-dependent kinase II activity by membrane voltage and by calcium influx. Proc Natl Acad Sci U S A 91, 9659-9663.
Yuan, W., and Bers, D. M. (1994). Ca-dependent facilitation of cardiac Ca current is due to Ca-calmodulin-dependent protein kinase. Am J Physiol 267, H982-993.
Yue, D., Herzig, S., and Marban, E. (1990). b-Adrenergic Stimulation of Calcium Channels Occurs by Potentiation of High-Activity Gating Modes. PNAS 87, 753-757.
Zhang, T., Johnson, E. N., Gu, Y., Morissette, M. R., Sah, V. P., Gigena, M. S., Belke, D. D., Dillmann, W. H., Rogers, T. B., Schulman, H., et al. (2002). The Cardiac-specific Nuclear δ_{B} Isoform of Ca²⁺/Calmodulin-dependent Protein Kinase II Induces Hypertrophy and Dilated Cardiomyopathy Associated with Increased Protein Phosphatase 2A Activity. J Biol Chem 277, 1261-1267.
Zühlke, R. D., Pitt, G. S., Deisseroth, K., Tsien, R. W., and Reuter, H. (1999). Calmodulin supports both inactivation and facilitation of L-type calcium channels. Nature 399, 159-162.
Zühlke, R. D., Pitt, G. S., Tsien, R. W., and Reuter, H. (2000). Ca²⁺-sensitive Inactivation and Facilitation of L-type Ca²⁺ Channels Both Depend on Specific Amino Acid Residues in a Consensus Calmodulin-binding Motif in the α_{1C} subunit. J Biol Chem 275, 21121-21129.
Zühlke, R. D., and Reuter, H. (1998). Ca²⁺-sensitive inactivation of L-type Ca²⁺ channels depends on multiple cytoplasmic amino acid sequences of the α_{1C} subunit. Proceedings of the National Academy of Sciences of the United States of America 95, 3287-3294.
Zygmunt, A. C., and Maylie, J. (1990). Stimulation-dependent facilitation of the high threshold calcium current in guinea-pig ventricular myocytes. J Physiol 428, 653-671.

1. A method for determining whether an agent inhibits binding between CaMKII and a calcium channel, comprising:
(a) contacting (i) CaMKII, (ii) the calcium channel or a fragment thereof comprising a portion whose amino acid sequence is TVGKF(Y/I) and (iii) the agent, under conditions which, in the absence of the agent, permit binding between CaMKII and the channel or fragment thereof;
(b) determining the amount of binding between CaMKII and the channel or fragment thereof in step (a); and
(c) comparing the amount of binding determined in step (b) with the amount of binding between CaMKII and the channel or fragment thereof in the absence of the agent, whereby a lower amount of binding in the presence of the agent indicates that the agent inhibits binding between CaMKII and the calcium channel.

2. The method of claim 1, wherein the calcium channel is an L-Type calcium channel.

3. The method of claim 1, wherein the calcium channel is a P/Q-Type calcium channel.

4. The method of claim 1, wherein in step (a), a fragment of the calcium channel is used.

5. The method of claim 1, wherein in step (a), the CaMKII is autophosphorylated.

6. The method of claim 1, wherein in step (a), the fragment of calcium channel comprises a portion whose amino acid sequence is TVGKFY.

7. The method of claim 1, wherein in step (a), the fragment of calcium channel is of an L-type calcium channel and comprises a portion whose amino acid sequence is that of the portion of the channel from amino acid reside 1639 to amino acid residue 1660.

8. The method of claim 1, wherein in step (a), the fragment of calcium channel is at least about 10, 20 or 50 amino acid residues in length.

9. The method of claim 1, wherein the agent is known to be a kinase inhibitor.

10. The method of claim 1, wherein the agent is a polypeptide.

11. The method of claim 10, wherein the agent is a polypeptide comprising a fragment of calcium channel comprising a portion whose amino acid sequence is TVGKF(Y/I).

12. The method of claim 11, wherein the agent is a polypeptide comprising a fragment of calcium channel comprising a portion whose amino acid sequence is TVGKFY.

13. The method of claim 10, wherein the agent is a polypeptide comprising a fragment of L-type calcium channel comprising a portion whose amino acid sequence is that of the portion of the channel from amino acid reside 1639 to amino acid residue 1660.

14. The method of claim 10, wherein the agent is a polypeptide comprising a fragment of calcium channel and is at least about 10, 20 or 50 amino acid residues in length.

15-25. (canceled)

26. A method for inhibiting binding between CaMKII and a calcium channel in a cell comprising contacting the cell with an agent that inhibits binding between CaMKII and an L-Type and/or P/Q-Type calcium channel.

27. A method for inhibiting binding between CaMKII and a calcium channel in a cell comprising contacting the cell with a polypeptide comprising a fragment of an L-Type calcium channel comprising a portion whose amino acid sequence is TVGKF(Y/I).

28. The method of claim 26 or 27, wherein the calcium channel is an L-type channel and the cell is a cardiac cell.

29. The method of claim 26 or 27, wherein the cell is a neuronal cell.

30-42. (canceled)


SEQUENCE LISTING (3 sequences)

SEQ ID NO: 1 - protein, 22 aa, Rat
KAQKKNRNKLRRQHSYDTFVDL

SEQ ID NO: 2 - protein, 22 aa, Rat
SHRSTVASCMHRQETVDCLKKF

SEQ ID NO: 3 - protein, 22 aa, Rat
GDDEVTVGKFYATFLIQEYFRK